(19) United States
(12) Patent Application Publication
(10) Pub. No.: US 2023/0058124 A1
(43) Pub. Date: Feb. 23, 2023

(54) PREECLAMPSIA BIOMARKERS AND USE THEREOF

(71) Applicant: INNOVATIO PTY LTD, Cremome NSW (AU)

(72) Inventor: Lana McClements, New South Wales (AU)

(21) Appl. No.: 17/758,083

(22) PCT Filed: Dec. 22, 2020
(86) PCT No.: PCT/AU2020/051416

§ 371 Date: Jun. 28, 2022

Related U.S. Application Data
(60) Provisional application No. 62/954,529, filed on Dec. 29, 2019.

(51) Int. Cl. G01N 33/68

(52) U.S. Cl. CPC G01N 33/689; G01N 2333/70585; G01N 2333/99; G01N 2800/368; G01N 2800/50

(57) ABSTRACT

Biomarkers and combinations thereof for determining risk and onset of preeclampsia, methods for their use and for diagnosis, prediction of risk, management, and treatment of preeclampsia in pregnant human females are provided.

### FIELD OF THE INVENTION

The present invention is directed to methods for determining risk for developing preeclampsia in a pregnant female human subject. In particular, the invention concerns biomarkers and combinations thereof for determining risk and onset of preeclampsia, methods for their use and for diagnosing, prediction of risk, management and treatment of preeclampsia in pregnant human females are provided.

### BACKGROUND OF THE INVENTION

The following discussion of the background art is intended to facilitate an understanding of the present invention only. The discussion is not an acknowledgement or admission that any of the material referred to is or was part of the common general knowledge as at the priority date of the application.

Hypertensive disorders of pregnancy are a leading cause of maternal and neonatal morbidity and mortality. Preeclampsia, one of the most dangerous hypertensive disorders of pregnancy, develops after 20 weeks' gestation and occurs in 2-8% of pregnancies. Preeclampsia is determined following evidence of high blood pressure (≥140/90 mm Hg) on two separate occasions and end organ involvement, most commonly renal or liver. The only effective treatment of established preeclampsia is delivery of the placenta, which necessitates delivery of the baby, frequently at preterm gestations, which in turn is accompanied by increased risk of neonatal complications and even death. Preeclampsia earlier that 34 weeks'gestation is considered “early onset” preeclampsia, and preeclampsia after 34 weeks gestation is considered “late onset” preeclampsia.

A number of risk factors have been identified as associated with development of preeclampsia including maternal obesity, nulliparity, multiple pregnancies, maternal age, long inter-pregnancy intervals, previous stillbirths and underlying maternal medical conditions such as diabetes mellitus (Burton et al, BMJ. (2019) 366:12381).

There is great interest in developing early biomarkers of preeclampsia, however, poorly understood pathogenesis has hindered the development of reliable biomarkers, particularly early in pregnancy.

Angiogenesis-related biomarkers (e.g. sFlt-1 and PIGF, galectin-3, VEGF, see U.S. Pat. No. 9,518,992; Zeisler et al, N Engl J Med. 2016; 374(1):13-2; Schlembach et al, 2018; 18(1);603; Figueira et al, Pregnancy Hypertens 2018; 13:30-36) are only indicative of risk a few weeks before the onset of the preeclampsia. Nevertheless, when possible, prediction of preeclampsia has been associated with improved pregnancy outcomes and has been demonstrated to be cost-effective, even a short time before the clinical onset of disease.

Other predictive algorithms have been used successfully early in pregnancy between 11 and 13 weeks of gestation based on maternal characteristics, uterine artery pulsatility index and angiogenic factors such as PIGF and PAPP-A (Rolnik D L, Wright D, Poon L C Y, et al. ASPRE trial: performance of screening for preterm pre-eclampsia. Ultrasound Obstet Gynecol. 2017; 50 (4): 492-495). However, these are mainly effective at predicting early-onset or preterm preeclampsia. Therefore, risk stratification strategies for late-onset preeclampsia as well as evolving preeclampsia between the second and third trimester of pregnancy, are lacking. This is particularly important as stratifying pregnancies at risk of preeclampsia has been associated with improved pregnancy outcomes (Poon L C, McIntyre H D, Hyett J A, da Fonseca E B, Hod M, FIGO Pregnancy and NCD Committee. The first-trimester of pregnancy: a window of opportunity for prediction and prevention of pregnancy complications and future life. Diabetes Res Clin Pract. 2018; 145: 20-30) and appears cost-effective, even a few weeks before the clinical onset of disease (Schlembach D, Hund M, Schroer A, Wolf C. Economic assessment of the use of the sFlt-1/PIGF ratio test to predict preeclampsia in Germany. BMC Health Serv Res. 2018; 18(1): 603).

Although low-dose aspirin has been effective at preventing early-onset preeclampsia (Rolnik D L, Wright D, Poon L C, et al. Aspirin versus placebo in pregnancies at high risk for preterm preeclampsia, N Engl J Med. 2017; 377(7): 613 — 622), the only treatment of established preeclampsia is delivery of the placenta, which necessitates delivery of the baby, frequently at preterm gestations.

FK506-binding protein like (FKBPL) is a peptidyl prolyl cis trans isomerase which belongs to the immunophilin protein group with important roles in vascular diseases. As a key protein which regulates angiogenesis, FKBPL plays an important role in both developmental and pathological angiogenesis and stem cell differentiation. Abrogation of angiogenesis by FKBPL is dependent upon binding to the cell surface receptor, CD44.

The role of CD44 in angiogenesis is also well established. The potential of FKBPL as a predictive and prognostic biomarker has been suggested for breast, ovarian and other cancers (see, for example, Nelson et al, Oncotarget, 2015; 6(14):12209-12223; Robson et al, Drug Discovery Today, 2012; 17(11-12):544-548; Annett et al British Journal of Cancer 2020; 122(3):361-371; McClements et al BMC Cancer 2019; 19(1): 351 and Valentine et al, Clinical Cancer Res 2011; 17(5):1044-1056), neurodegenerative disease (US Patent Publication 2014/0315736 to Rowan et al) and infertility (US Patent Publication 2010/0305082 to Downes et al). The present inventor\ (Dr. McClements) has recently identified FKBPL as a prognostic biomarker for pre-eclampsia (US Patent Publication 2019/02043335).

Additional relevant publications include, but are not limited to US Patent Publications 2010/0016173 to Nagalla et al and 2019/079097 to Cooper et al (serum biomarker arrays for preeclampsia); 2010/0075348 to Schulze-Forster et al (anti-endothelin-receptor antibody biomarker for preeclampsia); 2018/0074070 to Hansson et al (hemopexin and alpha-1-micro-globulin biomarkers for preeclampsia); 2019/0002981 to Ward et al (DNA biomarkers for preeclampsia); 2017/0315130 to Grobe et al (copeptin biomarker for preeclampsia); 2017/0097358 to Simon et al (annexin A2 biomarker for preeclampsia) and 2017/0315130 to Grobe et al (miRNA biomarkers for preeclampsia).

Additional relevant references include McKeen et al, 2011; 39(2):663-668; North et al, 2011; 342:d1875; Kenny et al, 2014; 64(3):644-652; Rana et al, 2014: 63(2):198-202 and Levine et al, 2004; 350(7):672-683.

There is a need in the art for methods for accurate, early detection of subjects at risk of, or already experiencing preeclampsia, before the onset of severe symptoms. It is an objective of the invention to overcome one or more problems foreshadowed by the prior art.

### SUMMARY OF THE INVENTION

The present invention is founded on the principal of general application that the FKBPL-CD44 pathway serves an important role in the pathogenesis of pre-eclampsia, which can be utilized for diagnostic and therapeutic purposes. FKBPL and CD44 are differentially secreted early in pregnancy in women who proceeded to develop pre-eclampsia. When the CD44/FKBPL ratio is combined with mean arterial blood pressure (MABP) the predictive power of the test can be increased to identify the risk of pre-eclampsia in pregnant women.

According to an aspect of the present invention there is provided a method of determining risk for developing preeclampsia in a pregnant female human subject, the method comprising detecting the level of CD44 in a biological sample of the subject using an agent binding CD44, wherein a level of the CD44 above a predetermined threshold in the sample is indicative of an increased risk for the subject to develop preeclampsia at or after gestational week 30.

According to embodiments of the present invention the sample is of a gestation week 20 and on.

According to embodiments of the present invention, the method further comprises repeating the detecting at least once in a sample from a later gestational week and comparing with earlier CD44 level, wherein an increase in the level of CD44 in the later detection relative to the earlier CD44 level is indicative of an increased risk for the subject to develop preeclampsia at or after gestational week 30, and wherein the later gestational week is no later than gestational week 30.

According to an aspect of the present invention there is provided a method of determining risk for developing preeclampsia in a pregnant female human subject, the method comprising:

a. detecting the level of CD44 and the level of FKBPL in a biological sample of the subject using an agent binding CD44 and an agent binding FKBPL,

b. determining the ratio of CD44/FKBPL in the sample,

c. wherein a ratio of CD44/FKBPL above a predetermined threshold in the sample is indicative of an increased risk for the subject to develop preeclampsia at or after gestational week 30.

According to embodiments of the present invention, the sample is of gestational weeks 15-30.

According to embodiments of the present invention, the method further comprises repeating the detecting the level of CD44 and FKBPL and determining the CD44/FKBPL ratio at least once in a sample from a later gestational week and comparing with earlier CD44/FKBPL ratio, wherein an increase in the CD44/FKBPL ratio in the later detection relative to the earlier CD44/FKBPL ratio is indicative of an increased risk for the subject to develop preeclampsia at or after gestational week 30, and wherein the later gestational week is no later than gestational week 30.

According to embodiments of the present invention, the method further comprises determining MABP in the subject, and wherein elevated MABP and a ratio of CD44/FKBPL above a predetermined threshold in the sample is indicative of an increased risk for the subject to develop preeclampsia at or after gestational week 30.

According to an aspect of the present invention there is provided a method for managing pregnancy in a pregnant female human subject at gestational week 20-30, the method comprising:

a. detecting the level of CD44 in a biological sample of the subject using an agent binding CD44,

b. wherein a level of the CD44 is above a predetermined threshold in the sample, referring the subject for high risk pregnancy management.

According to an aspect of the present invention there is provided a method for managing pregnancy in a pregnant female human subject at gestational week 15-30, the method comprising:

a. detecting the level of CD44 and the level of FKBPL in a biological sample of the subject using an agent binding CD44 and an agent binding FKBPL,

b. determining the ratio of CD44/FKBPL in the sample,

c. wherein a ratio of CD44/FKBPL is above a predetermined threshold in the sample, referring the subject for high risk pregnancy management.

According to embodiments of the present invention, the method further comprises determining MABP in the subject and referring the subject for high-risk pregnancy management when MABP is elevated and the ratio of CD44/FKBPL is above the predetermined threshold.

According to embodiments of the present invention, the high risk pregnancy management comprises monitoring blood pressure and proteinuria in the subject.

According to embodiments of the present invention, the subject exhibits normal values for at least one indicator of preeclampsia selected from the group consisting of blood pressure, proteinuria and liver function.

According to an aspect of the present invention there is provided a method of treating a pregnant female human subject for preeclampsia, the method comprising:

a. detecting the level of CD44 in a biological sample of the subject using an agent binding CD44, wherein the biological sample is of gestational week 20-30,

b. wherein a level of the CD44 in the biological sample is above a predetermined threshold, testing the subject for at least one indicator of preeclampsia, and

c. wherein the at least one indicator of preeclampsia is positive, treating the subject for preeclampsia at or after gestational week 30.

According to embodiments of the present invention, step (a) further comprises determining MABP in the subject, and step (b) comprises testing the subject wherein levels of the CD44 and the MABP are above a predetermined threshold, and wherein the at least one indicator of preeclampsia is not MABP.

According to an aspect of the present invention there is provided a method of treating a pregnant female human subject for preeclampsia, the method comprising:

a. detecting the level of CD44 and a level of FKBPL in a biological sample of the subject using an agent binding CD44 and an agent binding FKBPL, wherein the biological sample is of gestational week 15-30,

b. wherein a ratio of the CD44/FKBPL levels in the biological sample is above a predetermined threshold, testing the subject for at least one indicator of preeclampsia, and

c. wherein the at least one indicator of preeclampsia is positive, treating the subject for preeclampsia at or after gestational week 30.

According to an aspect of the present invention there is provided a method of treating a pregnant female human subject for preeclampsia, the method comprising:

a. detecting the level of CD44 and a level of FKBPL in a first biological sample of the subject using an agent binding CD44 and an agent binding FKBPL, wherein the first biological sample is of gestational week 15-30,

b. detecting the level of CD44 and a level of FKBPL in a second biological sample of the subject using an agent binding CD44 and an agent binding FKBPL, wherein the second biological sample is of gestational week later than gestational week 30,

c. wherein a ratio of the CD44/FKBPL levels in the first biological sample is above a first predetermined threshold and a ratio of the CD44/FKBPL levels in the second biological sample is below a second predetermined threshold, testing the subject for at least one indicator of preeclampsia, and

d. wherein the at least one indicator of preeclampsia is positive, treating the subject for preeclampsia.

According to embodiments of the present invention, step (a) further comprises determining MABP in the subject, and step (b) comprises testing the subject wherein the ratio of the CD44/FKBPL levels and the MABP are above a predetermined threshold, and wherein the at least one indicator of preeclampsia is not MABP.

According to embodiments of the present invention, the at least one indicator is selected from the group consisting of hypertension, proteinuria, low platelet count, impaired liver function, signs of kidney problems (other than proteinuria), pulmonary edema and headaches or visual disturbances.

According to embodiments of the present invention, the preeclampsia treatment is selected from the group consisting of anti-hypertensive treatment, steroids and seizure prophylaxis.

According to an aspect of the present invention there is provided a method of treating a pregnant female human subject for preeclampsia, the method comprising:

a. detecting the level of CD44 in a biological sample of the subject using an agent binding CD44, wherein the biological sample is of gestational week later than gestational week 30,

b. wherein a level of the CD44 in the biological sample is below a predetermined threshold, testing the subject for at least one indicator of preeclampsia, and

c. wherein the at least one indicator of preeclampsia is positive, treating the subject for preeclampsia.

According to embodiments of the present invention, the at least one indicator of preeclampsia is MABP.

According to an aspect of the present invention there is provided a method of treating a pregnant female human subject for preeclampsia, the method comprising:

a. detecting the level of CD44 and a level of FKBPL in a biological sample of the subject using an agent binding CD44 and an agent binding FKBPL, wherein the biological sample is of a gestational week later than gestational week 30,

b. wherein a ratio of the CD44/FKBPL levels in the biological sample is below a predetermined threshold, testing the subject for at least one indicator of preeclampsia, and

c. wherein the at least one indicator of preeclampsia is positive, treating the subject for preeclampsia.

According to embodiments of the present invention, the at least one indicator is selected from the group consisting of hypertension, proteinuria, platelet count, impaired liver function, signs of kidney problems (other than proteinuria), pulmonary edema and headaches or visual disturbances.

According to embodiments of the present invention, the at least one indicator of preeclampsia is not MABP.

According to embodiments of the present invention, the preeclampsia treatment is selected from the group consisting of anti-hypertensive treatment, steroids and seizure prophylaxis.

According to embodiments of the present invention, the biological sample is whole blood or an isolated blood fraction.

According to embodiments of the present invention, the isolated blood fraction is serum or plasma.

According to embodiments of the present invention, the agent binding CD44 is an antibody specifically binding CD44.

According to embodiments of the present invention, the agent binding FKBPL is an antibody specifically binding FKBPL.

According to embodiments of the present invention, the antibody is a monoclonal antibody.

According to embodiments of the present invention, the predetermined threshold is a level of the CD44 or the ratio of CD44/FKBPL in normal, healthy pregnant gestational age-matched females.

According to an aspect of the present invention there is provided a composition-of-matter comprising a biological sample of a pregnant female human subject comprising an agent binding CD44 and an agent binding FKBPL.

According to an aspect of the present invention there is provided a composition of matter comprising:

a. a first portion of a biological sample of a pregnant female human subject comprising an agent binding CD44, and

b. a second portion of the biological sample of the pregnant female human subject comprising an agent binding FKBPL.

According to embodiments of the present invention, the biological sample is whole blood or an isolated blood fraction.

According to embodiments of the present invention, the isolated blood fraction is plasma or serum.

According to embodiments of the present invention, the sample is of a gestational week of 15 weeks and on.

According to embodiments of the present invention, the agent binding CD44 is an antibody specifically binding CD44.

According to embodiments of the present invention, the agent binding FKBPL is an antibody specifically binding FKBPL.

According to embodiments of the present invention, the antibody is a monoclonal antibody.

According to an aspect of the present invention there is provided a diagnostic kit for use in diagnosing preeclampsia or risk to develop preeclampsia in a pregnant female human subject, the kit comprising: (a) a first agent which specifically binds soluble CD44, wherein the first agent is detectably labeled or coupled to an enzyme; (b) a normal reference sample for CD44, wherein the normal reference sample for CD44 is a sample containing a level of CD44 found in a pregnant human female subject not having preeclampsia or risk to develop preeclampsia; a pregnant human female subject not having early-onset preeclampsia or eclampsia; (c) a second agent which binds soluble FKBPL wherein the second agent is detectably labeled or coupled to an enzyme and (d) a normal reference sample for FKBPL, wherein the normal reference for FKBPL sample is a sample containing a level of FKBPL found in a pregnant human female subject not having preeclampsia or risk to develop preeclampsia; a pregnant human female subject not having early onset preeclampsia.

According to embodiments of the present invention, the normal reference sample of CD44 contains CD44 in a range of concentrations between 20 and 300 ng/ml.

According to embodiments of the present invention, the normal reference sample of FKBPL contains FKBPL in a range of concentrations between 0.1 and 5 ng/ml.

According to embodiments of the present invention, agent binding FKBPL is an antibody, or FKBPL-binding fragment thereof.

According to embodiments of the present invention, the agent binding CD44 is an antibody, or CD44-binding fragment thereof.

According to embodiments of the present invention, the antibody is a monoclonal antibody.

According to embodiments of the present invention, the agent binding CD44 and/or the agent binding FKBPL are immobilized in a solid phase.

According to an aspect of the present invention there is provided an agent for treatment of preeclampsia for use in a pregnant human female subject exhibiting a level of CD44:

a. greater than a predetermined threshold in a biological sample therefrom, wherein the sample is from 20-30 weeks gestation; or

b. lower than a second predetermined threshold, wherein the sample is from 30 or more weeks gestation.

According to an aspect of the present invention there is provided an agent for treatment of preeclampsia for use in a pregnant human female subject exhibiting a ratio of CD44/FKBPL:

a. greater than a predetermined threshold in a biological sample therefrom, wherein the sample is from 15-30 weeks gestation, or

b. lower than a second predetermined threshold wherein the sample is from 30 or more weeks gestation.

According to embodiments of the present invention, the pregnant human female subject further exhibits an elevated MABP.

According to embodiments of the present invention, the biological sample is whole blood or an isolated blood fraction.

According to embodiments of the present invention, the isolated blood fraction is plasma or serum.

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### BRIEF DESCRIPTION OF THE SEVERAL VIEWS OF THE DRAWINGS

Some embodiments of the invention are now described, by way of example only, with reference to the following accompanying drawings.

FIGS. 1A-1D are graphs showing the correlation between plasma FKBPL and CD44 and the incidence of preeclampsia: FIG. 1A shows significant elevation of plasma CD44 levels at 20 weeks gestation (n=62, preeclampsia; n=60, controls) but none at 15 weeks gestation, in women who later developed preeclampsia (n=60) compared to healthy controls (n=60); two-tailed unpaired T-test, *p=0.02. In contrast, plasma FKBPL levels were reduced at both 15 and 20 weeks gestation in women who developed preeclampsia (n=61, 15 weeks; n=57, 20 weeks) compared to healthy controls (n=59, 15 weeks; n=58, 20 weeks); Mann-Whitney test, * p=0.03 (15 weeks); * p=0.01 (20 weeks) (FIG. 1B). FIG. 1C shows that the CD44/FKBPL ratio is elevated in women who proceeded to develop preeclampsia. For each patient, CD44 plasma levels were divided by FKBPL plasma levels to obtain the ratio at each time point. Preeclampsia (n=57, 15 weeks; n=54, 20 weeks) compared to healthy controls at the same gestation age (n=56, 15 weeks; n=55, 20 weeks); Mann-Whitney test, *p=0.02 (15 weeks); **p=0.004 (20 weeks). FIG. 1D is the Receiver operating characteristics (ROC curve) for the CD44/FKBPL ratio for prediction of preeclampsia; AUC: area under the curve. Graphs 1A-1C: Data were presented as means±SEM. *<0.05, **<0.01;

FIGS. 2A-2D are scatter plots showing the change in FKBPL and CD44 levels with onset of preeclampsia: FKBPL (FIG. 2A) and CD44 (FIG. 2B) were measured in plasma from pregnant women post preeclampsia diagnosis (n=18) and compared to healthy pregnant controls (n=14). Note the increase in serum FKBPL (FIG. 2A, PE) and decrease in CD44 (FIG. 2B, PE), compared to controls. CD44/FKBPL ratio (FIG. 2C, PE) is decreased, compared to controls, in the pregnant women post preeclampsia diagnosis. Secreted FKBPL levels were higher in cultured adipose mesenchymal stem cell (MSC) from women diagnosed with preeclampsia vs. controls (FIG. 2D, MSC-PE, n=3). Statistical calculations according to two-tailed unpaired t-test. FKBPL secretion in preeclampsia was adjusted to the number of MSC; two-tailed unpaired t-test. Data were presented as mean±SEM; *<0.05, ***<0.001.

FIGS. 3A-3C are graphs showing the effect of hypoxia and MSC on angiogenic properties of HUVECs in association with reduced FKBPL and increased CD44 levels: HUVEC differentiation was evaluated using a tubule formation assay; cells were stained with calcein and trypsinised before plating 50,000 cells in Matrigel and exposing them to hypoxia (1% O2) or normoxia (21% O2) for 6 hours in complete or mesenchymal stem cells-conditioned medium (MSC-CM) (n=6). Cells were imaged using a Leica DMi8 fluorescent microscope and the length of tubules formed quantified using ImageJ software (FIG. 3A). Protein lysates were collected from HUVECs exposed to hypoxia (1% O2) or normoxia (21% O2) in complete medium or MSC-CM for 24 h, and Western blotting performed to determine FKBPL (42 kDa) and GAPDH (37kDa) protein expressions (n=3). FKBPL protein expression was quantified using ImageJ and adjusted to GAPDH expression (FIG. 3B). Real-time qPCR analyses of FKBPL and CD44 mRNA levels relative to the 18S housekeeping gene were carried out using HUVECs exposed to complete or MSC-CM in hypoxia for 24 h (n=3). Serum free medium was used as a negative control (FIG. 3C). Results are expressed as mean±SEM; statistical analyses were performed using two-way ANOVA with Sidak's multiple comparisons test. *<0.05, **<0.01. MSC-CM: mesenchymal stem cell—conditioned medium (FIG. 3C);

FIGS. 4A-4D are graphs showing that trophoblast cell migration is stimulated by hypoxia or MSC treatment and hypoxia regulated FKBPL and CD44: Migration of BeWo (FIG. 4A) or Jar (FIG. 4B) cells was evaluated using a wound healing-scratch assay, in hypoxia (1% O2) or normoxia (21% O2), treated with complete cultivation medium or MSC-CM for 24 h (n=3). FIG. 4C shows the Real-Time qPCR analysis of FKBPL and GAPDH mRNA levels (n=3) relative to the 18S housekeeping gene in BeWo trophoblast cells following exposure to MSC-CM for 24 hours in hypoxia (1% O2). FIG. 4D shows the Real-time qPCR analyses of FKBPL and CD44 mRNA levels (n=3) relative to the 18S housekeeping gene in Jar trophoblast cells following exposure to MSC-CM for 24 hours in hypoxia (1% O2). Results are expressed as mean±SEM; statistical analyses were performed using two-way ANOVA with Sidak's or Tukey's multiple comparisons test. *<0.05, **<0.01 and ***<0.001. MSC-CM: mesenchymal stem cell-conditioned medium

### SEQUENCE LISTINGS

The ASCII file, entitled 77958 _BRH_ST25.txt, created on Dec. 22, 2020, submitted concurrently with the filing of this application is incorporated herein by reference. The sequence listings being:

| | | | |
|---|---|---|---|
| SEQ ID NO: 1 | DNA | Homo | Nucleic acids encoding the human |
| | | sapiens | FKBPL polypeptide |
| SEQ ID NO: 2 | PRT | Homo | Polypeptide of FKBPL, GenBank |
| | | sapiens | Accession number NP_079313 |
| SEQ ID NO: 3 | DNA | Homo | Nucleic acids encoding the human |
| | | sapiens | CD44 polypeptide |
| SEQ ID NO: 4 | PRT | Homo | Polypeptide of CD44, GenBank |
| | | sapiens | Accession number XP_005253288.1 |
| SEQ ID NO: 5 | PRT | Artificial | Biotin lygase tag amino acid |
| | | Sequence | sequence |

### DETAILED DESCRIPTION OF EMBODIMENTS OF THE INVENTION

The present invention, in some embodiments thereof, relates to markers and methods for early determination of risk of developing preeclampsia in pregnant females, methods for monitoring pregnant females at-risk for preeclampsia, and methods for selecting management and treating pregnant females with or at risk for preeclampsia.

For convenience, the following section generally outlines various definitions of terms used herein. Following that discussion, the description presents illustrative embodiments of the invention followed by specific examples demonstrating the properties of various embodiments of the invention and how they can be employed. The illustrative embodiments of the invention are not limited in application to the details set forth in the following description or as exemplified by the Examples. The invention is capable of other embodiments or of being practiced or carried out in various ways.

### 1. General Definitions

Those skilled in the art will appreciate that the invention described herein is susceptible to variations and modifications other than those specifically described. The invention includes all such variations and modifications. The invention also includes all the steps, features, formulations and sequences referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however, be understood to be common general knowledge.

Manufacturer's instructions, descriptions, product specifications, and product sheets for any products mentioned herein or in any document incorporated by reference herein, are hereby incorporated herein by reference, and can be employed in the practice of the invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Other than in the operating examples, or where otherwise indicated, all numbers expressing quantities of ingredients or reaction conditions used herein should be understood as modified in all instances by the term “about.” The term “about” when used in connection with percentages can mean±10%.

The invention described herein may include one or more range of values (e.g. size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this specification, the use of the singular also includes the plural unless specifically stated otherwise. In this application, the use of “or” means “and/or” unless stated otherwise. Furthermore, the use of the term “including”, as well as other forms, such as “includes” and “included”, is not limiting.

Throughout this specification, unless the context requires otherwise, the word “comprise” or variations such as “comprises”, “comprising”, “includes”, “including” or “having” will be understood to imply the inclusion of a stated integer or group of integers but not the exclusion of any other integer or group of integers. That is they mean “including but not limited to”. The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### 2. Illustrative Embodiments

Currently available biomarkers for preeclampsia are of limited value for detecting preeclampsia, particularly late-onset preeclampsia phenotype. FK506-binding protein like (FKBPL) is a peptidyl prolyl cis trans isomerase of the immunophilin protein group that has recently been identified as a prognostic biomarker for preeclampsia (US 2019/0305082 to McClements). The present inventor has surprisingly discovered that changes in levels of CD44, the cell surface receptor for FKBPL, is a reliable indication of risk of preeclampsia as early as gestational week 20, and that combining FKBPL and CD44 (CD44/FKBPL ratio) provides a sensitive, early indicator of risk for developing preeclampsia in pregnant women, effective as early as gestational week 15. The inventors have further uncovered that combining the CD44/FKBPL ratio with assessment of mean arterial blood pressure (MABP) provides increased accuracy for early prediction of risk of preeclampsia, significantly extending the ranges of Odds Ratio for preeclampsia.

Extending her observations to the later stages of pregnancy (e.g., third trimester), the inventor has discovered a significant reversal of the trend in levels of CD44 and CD44/FKBPL ratio in pregnant females diagnosed with preeclampsia, after week 30, affording a previously unavailable tool for monitoring and managing at-risk pregnancies as well as for determining onset of disease.

Thus, in one aspect of the present invention there is provided a method of determining risk for developing preeclampsia in a pregnant female human subject, the method comprising detecting the level of CD44 in a biological sample of the subject using an agent binding CD44, wherein a level of the CD44 above a predetermined threshold in the sample is indicative of an increased risk for the subject to develop preeclampsia. In specific embodiments, the increased risk is for developing preeclampsia at or after gestational week 30.

In another aspect of the present invention, there is provided a method for determining risk for developing preeclampsia in a pregnant female human subject, the method comprising detecting the level of CD44 and the level of FKBPL in a biological sample of the subject using an agent binding CD44 and an agent binding FKBPL, determining the ratio of CD44/FKBPL in the sample, wherein a ratio of CD44/FKBPL above a predetermined threshold in the sample is indicative of an increased risk for the subject to develop preeclampsia. In specific embodiments, the increased risk is for developing preeclampsia at or after gestational week 30.

In addition to CD44 levels and the CD44/FKBPL ratio, maternal blood pressure can be included as a variable useful for determining risk of the subject to develop preeclampsia. Thus, in some embodiments, the method of the invention further comprises determining MABP in the subject, whereas elevated MABP and elevated CD44 levels and/or elevated CD44/FKBPL above a predetermined threshold is indicative of an increased risk for the subject to develop preeclampsia. Methods for measuring blood pressure, and ranges of normal blood pressure values throughout pregnancy are well known. In specific embodiments, “normal blood pressure” is defined as MABP equal to or lower than 85 mm Hg, and “elevated blood pressure” is defined as MABP greater than 85 mm Hg.

As used herein, the term “preeclampsia” or “pre-eclampsia” refers to a multisystem complication of pregnancy that may be accompanied by one or more of high blood pressure, proteinuria, swelling of the hands and face/eyes (edema), headache, sudden weight gain, higher-than-normal liver enzymes, and thrombocytopenia. Preeclampsia typically occurs in the third trimester of pregnancy, but in severe cases, the disorder occurs in the second trimester, e.g., after about the 20th week of pregnancy. If left unaddressed, preeclampsia can lead to eclampsia, i.e., seizures that are not related to a preexisting brain condition.

By “determining the risk for developing preeclampsia”, or “prognosing” a preeclampsia, or “providing a preeclampsia prognosis,” it is generally meant providing a preeclampsia prediction, e.g., a prediction of a subject's susceptibility, or risk, of developing preeclampsia; a prediction of the course of disease progression and/or disease outcome, e.g. expected onset of the preeclampsia, expected duration of the preeclampsia.

As used herein, the term “developing preeclampsia” refers to suffering from sufficient clinical indicators to be formally diagnosed with preeclampsia. Detailed description of clinical indicators of preeclampsia is provided below.

In some embodiments, the level of CD44 or CD44/FKBPL ratio of the subject's biological sample will be indicative of the increased risk of developing preeclampsia in mid-late gestation. In specific embodiments, the risk is risk of developing preeclampsia between gestational weeks 20-30. In other embodiments, the risk of developing preeclampsia is risk of developing preeclampsia at or after gestational week 30. In still other embodiments, the risk of developing preeclampsia is risk of developing preeclampsia between gestational weeks 30-35, e.g., gestational week 30, 31, 32, 33, 34 or 35.

As used herein, “diagnosing” a preeclampsia or “providing a preeclampsia diagnosis,” generally refers to providing a preeclampsia determination, e.g. a determination as to whether a subject (e.g. a subject that has one or more clinical symptoms of preeclampsia, a subject that is asymptomatic for preeclampsia but has risk factors associated with preeclampsia, a subject that is asymptomatic for preeclampsia and has no risk factors associated with preeclampsia) is presently affected by preeclampsia; a classification of the subject's preeclampsia into a subtype of the disease or disorder; a determination of the severity of preeclampsia; and the like.

The normal serum concentration of CD44 is approximately 100-135 ng/mL during mid-pregnancy, with an average of 115 ng/mL. In some embodiments, a level of CD44 greater than 120 ng/ml, greater than 125, 130, 135, 140, 150, 175, 200, 225, 250, 275, 300, 325, 350, 375, 400 or more ng/ml in mid-pregnancy is indicative of an increased risk of developing preeclampsia. In specific embodiments, CD44 levels in the range of 120-140 ng/ml, e.g., greater than 125 ng/ml at mid-pregnancy (e.g., gestational age 15-20 weeks), are indicative of increased risk for preeclampsia. Normal serum concentration of FKBPL is approximately 0.8-1.15 ng/ml during mid-pregnancy, with an average of 1.0 ng/mL at 15 weeks and 0.89 ng/mL at 20 weeks. In some embodiments, the normal mid-pregnancy serum CD44/FKBPL ratio is in the range of 125-165, with an average of 129 at 15 weeks and 143 at 20 weeks gestation, and thus, a CD44/FKBPL ratio greater than 140, greater than 145, 150, 155, 160, 170, 180, 190, 200, 225, 250, 275, 300, 325, 350, 375, 400 or more in mid-pregnancy is indicative of an increased risk of developing preeclampsia. In specific embodiments, CD44/FKBPL ratios in the range of 140-200, e.g., greater than 140 at mid-pregnancy (e.g., gestational age 15 weeks) or 150 (gestational age 20 weeks), are indicative of increased risk for preeclampsia.

In later gestation, mean normal serum concentration of CD44 observed was approximately 105 ng/mL while CD44 levels for women with diagnosed preeclampsia was consistently lower, approximately 85 ng/mL on the average. Thus, in some embodiments, a level of CD44 after week 30, lower than 100, lower than 90, 85, 80 or less ng/ml in mid-pregnancy, is an indicator for further testing for at least one indicator of preeclampsia for determining treatment. In other embodiments, the average normal later pregnancy serum CD44/FKBPL ratio is 136 ng/mL at greater than 30 weeks gestation, while the serum CD44/FKBPL ratio for women diagnosed with preeclampsia was approximately 105. Thus, in some embodiments, a CD44/FKBPL ratio lower than 135, lower than 130, 125, 120, 110, 105, 100, 90, 80, 70 or less after gestational week 30 is an indicator for further testing for at least one indicator of preeclampsia for determining treatment.

It will be noted that ranges of values (CD44, FKBPL, CD44/FKBPL) within the risk and normal pregnant female populations may overlap, being representative of a distribution of individual observations. Thus, in some embodiments, wherein a subject's biomarker (e.g. CD44, FKBPL, CD44/FKBPL) levels indicate elevated risk, but also fall within the overlap between risk and normal levels, corroboration of the elevated risk, strategy for management of pregnancy, monitoring and/or referral for treatment should be sought, for example, by testing or referral for testing of at least one indicator of preeclampsia (e.g., blood pressure, kidney function, liver function, proteinuria, etc).

Typically, but not exclusively, the greater the divergence of the subject's CD44 levels and/or CD44/FKBPL ratio (and, where included, the MABP), from the corresponding values from samples from normal healthy pregnant females, the stronger the indication of risk to develop or, if assessing later gestational age, actual presence of preeclampsia.

Further, as mentioned above, reduced CD44 levels and/or CD44/FKBPL ratio, in later gestation, compared to CD44 levels or CD44/FKBPL ratio first assayed earlier in mid-gestation can be a strong indicator for further testing for preeclampsia indicators, and initiation, if needed, of treatment. It will be noted that subjects having had mid-gestational CD44 levels and/or CD44/FKBPL ratios indicative of increased risk of developing preeclampsia, and subsequent reduced CD44 levels and/or CD44/FKBPL ratios after gestational week 30 should be considered candidates for further testing for active preeclampsia and suitable treatment.

In other embodiments, a threshold of CD44 concentration and/or CD44/FKBPL ratio is determined, and increased CD44 and/or CD44/FKBPL ratio above the predetermined threshold is indicative of increased risk to develop preeclampsia.

The predetermined threshold can be a value or range of values outside the range of CD44 concentrations and/or CD44/FKBPL ratios determined for normal, healthy pregnant human females, or may be a value of CD44 concentration and/or CD44/FKBPL ratio assayed in a sample or samples taken from matched subject or subjects (e.g. matched for at least one of the following criteria: gestational age of the fetus, age of the mother, blood pressure prior to pregnancy, blood pressure during pregnancy, BMI of the mother, weight of the fetus, prior diagnosis of preeclampsia or eclampsia, and a family history of preeclampsia or eclampsia, number of prior pregnancies, disease and other clinical parameters) who do not have preeclampsia or high risk for preeclampsia. The reference sample for determining the threshold can also be a prior sample taken from the same subject (as in monitoring).

In some embodiments, risk for developing preeclampsia, or risk for developing preeclampsia at or after gestational week 30 is expressed in fold increase relative to preeclampsia risk in a normal, healthy pregnant female. Thus, the risk for developing preeclampsia determined according to the methods of the invention can be, in some cases, mildly elevated (e.g., 1.2, 1.4, 1.5, 1.6) relative to that of normal healthy pregnant females, while significantly elevated CD44 levels (at 20-30 weeks gestation) or elevated CD44/FKBPL ratio (at 15-30 weeks gestation) can indicate risk increased between 1.5 to 5-fold, 1.5, 2.0, 2.5, 3.0, 3.5, 4.0, 4.5 or 5.0 fold that of normal health pregnant females. In subjects having the additional risk factor of high blood pressure (MABP >85 mmHg), the convergence of factors and indicators can indicate risk increased greater than 10-fold the risk determined for normal healthy pregnant females.

The method of determining risk of developing preeclampsia in the subject according to some embodiments of the invention enables the classification of a subject as a high risk or low risk pregnancy, and referral for high-risk pregnancy management. Typically, a high-risk pregnancy management protocol will include scheduling a greater number of prenatal office visits with an obstetrician, special attention to maternal health, eating a nutritious diet, monitoring weight gain, proteinuria and blood pressure regularly, symptoms of preeclampsia such as headache, avoidance of risky substances, food or medications, vitamins and iron supplements or pre-natal health medication or supplements. In addition to regular screening exams, additional tests may be recommended to further assess the health and development of the baby. These may include a biophysical profile or targeted ultrasound. Further, delivery of a high-risk pregnancy is recommended to take place in a hospital setting rather than at home. Depending on the individual case, the baby may be delivered vaginally or through a Cesarean-section (C-section). In specific embodiments, high risk management comprises frequent monitoring of blood pressure and proteinuria in the subject.

Since the responsiveness to preeclampsia management and treatment may affect disease outcome, the teachings of the invention can be used to determine the prognosis of a subject in need of pregnancy management. In this regard it is important to note that proper management of preeclampsia can be critical for allowing at-risk pregnancies to extend beyond 37 weeks, preventing premature birth. Thus, in some embodiments, the methods of the present invention can be used to prevent premature birth. In particular, the present methods of evaluated risk or presence of preeclampsia, can be used to provide pregnant females with management and/or treatment delaying or obviating the need for premature delivery of the fetus.

According to some embodiments of the invention, the method further comprises informing the subject of the predicted risk of preeclampsia and/or the predicted prognosis of the subject.

As used herein the phrase “informing the subject” refers to advising the subject that based on the levels of CD44 or CD44/FKBPL ratio, the subject should seek a suitable regimen of management and/or treatment. For example, if the subject is predicted to respond to anti-hypertensive therapy and anti-coagulation therapy and is diagnosed or suffers from a pathology requiring that such a treatment is advisable.

Once the level of CD44 or CD44/FKBPL ratio is determined, the results can be recorded in the subject's medical file, which may assist in selecting a treatment regimen and/or determining prognosis of the subject.

According to some embodiments of the invention, the method further comprises recording the levels of CD44 or CD44/FKBPL ratio of the subject, and/or the level of risk of preeclampsia in the subject's medical file.

As mentioned, the prediction of the risk of a subject to develop preeclampsia can be used to select the treatment regimen of a subject and thereby treat the subject in need thereof. Thus, according to some aspects of the invention, there is provided a method of treating a pregnant female human subject for preeclampsia, the method comprising detecting the level of CD44 in a biological sample of the subject using an agent binding CD44, wherein when the level of CD44 in the biological sample is above a predetermined threshold, testing the subject for at least one indicator of preeclampsia, and if the at least one indicator of preeclampsia is positive, treating the subject for preeclampsia. In specific embodiments, the biological sample is of gestational week 20-30.

In other embodiments, there is provided a method of treating a pregnant female human subject for preeclampsia, the method comprising detecting the level of CD44 and FKBPL in a biological sample of the subject using an agent binding CD44 and an agent binding FKBPL, wherein when the ratio CD44/FKBPL in the biological sample is above a predetermined threshold, testing the subject for at least one indicator of preeclampsia, and if the at least one indicator of preeclampsia is positive, treating the subject for preeclampsia. In specific embodiments, the biological sample is of gestational week 15-30.

In some embodiments, the at least one indicator of preeclampsia is any one of clinically recognized indicators, for example, as detailed herein. In other embodiments, in addition to CD44 levels and the CD44/FKBPL ratio, maternal blood pressure can be included as a variable useful for determining treatment of preeclampsia. Thus, in some embodiments, the method of the invention further comprises determining mean arterial blood pressure (MABP) in the subject, and wherein the MABP is elevated, CD44 levels and/or CD44/FKBPL are elevated above a predetermined threshold and the at least one indicator of preeclampsia, which is not MABP, is positive, treating the subject for preeclampsia.

Typically, preeclampsia is definitively diagnosed in late-middle to late pregnancy, however, cases of early-onset preeclampsia, as early as 20 weeks have been reported. Thus, although treatment can be initiated where necessary at any time following determining of CD44 and/or CD44/FKBPL ratio and testing at least one preeclampsia indicator, in some embodiments treating the subject for preeclampsia is affected at or after gestational week 30. In other embodiments, treating the subject for preeclampsia is affected at or after gestational week 20, at gestational week 21, 22, 23, 24, 25, 26, 27, 30, 31, 32, 33, 34, 35, 36, 37, 38, 39 or 40 or more.

Repeated determination of CD44 and FKBPL levels can be useful for monitoring changes in the risk of the pregnant female subject for developing preeclampsia. Thus, in one embodiment, the method further comprises repeating the detection of CD44 and/or FKBPL at least once in a sample of the subject from a gestational week later than the first sample, and comparing the level of CD44 and/or CD44/FKBPL ratio with the earlier CD44 level or CD44/FKBPL ratio, whereas an increase in the level of CD44 or the CD44/FKBPL ratio in the later detection, relative to the earlier CD44 level and/or earlier CD44/FKBPL ratio is indicative of an increased risk for the subject to develop preeclampsia. In specific embodiments, the increased risk is for developing preeclampsia at or after gestational week 30. In other embodiments, the sample of the subject from a later gestational week is a sample from a gestational week no later than week 30.

Repeat determination of CD44 and FKBPL levels can be performed at any time following an initial determination. As determined by the subject's medical personnel (e.g., obstetrician, GP), repeat determination can be performed at regular intervals following the initial assessment of CD44 and FKBPL levels, or, alternatively or in addition, in response to changes (or lack thereof) in the character, severity and/or frequency of preeclampsia indicators. In some embodiments, repeat determination of CD44 and/or FKBPL levels can be performed days after the initial determination, e.g., 1, 2, 3, 4, 5, 6, 7 days afterwards, or 1, 1.5, 2, 2.5, 3, 3.5, 4, 4.5, 5, 5.5, 6, 6.5, 7, 7.5, 8, 8.5, 9 or more weeks after the initial determination of CD44 and/or CD44/FKBPL ratio in the subject's sample. In a specific embodiment, repeat determination of CD44 and/or CD44/FKBPL is performed for a sample of gestational week 15-30, one week following the initial determination. In other embodiments, repeat determination of CD44 and/or CD44/FKBPL is performed for a sample of gestational week 2 or 3 weeks following the initial determination.

It will be appreciated that repeat determination of CD44 levels and/or CD44/FKBPL ratio and comparison with values from normal pregnancies, or with CD44 levels and/or CD44/FKBPL ratio can be performed more often than once, and that multiple assessments of CD44 levels and/or FKBPL levels in the subject's samples from different time points can be beneficial in determining further risk of the subject developing preeclampsia as well as aiding in strategizing the monitoring of the patient's preeclampsia indicators.

As used herein, the term “monitoring” a preeclampsia generally refers to monitoring, and in particular, recording changes in a subject's condition, e.g., to inform a preeclampsia diagnosis, to inform a preeclampsia prognosis, to provide information as to the effect or efficacy of a preeclampsia treatment, and the like.

Investigating the levels of CD44 and FKBPL in later gestation, in healthy pregnant women and women diagnosed with preeclampsia, the instant inventor has surprisingly uncovered significant changes in the concentrations of the biomarkers with the onset of preeclampsia, relative to the values in normal, healthy pregnancies, with CD44 concentration dropping relative to normal and FKBPL concentration increasing relative to normal. This previously unrecognized and surprising reversal in the trends of the biomarker's relative concentrations can be used to indicate onset of preeclampsia, and to determine treatment and management of pregnant women in later stages of pregnancy before the onset of severe symptoms and pregnancy complications.

Thus, according to a further aspect of the invention there is provided a method of treating a pregnant female human subject for preeclampsia, the method comprising detecting the level of CD44 in a biological sample of the subject using an agent binding CD44, wherein the biological sample is of gestational week later than week 30, wherein the level of CD44 in the biological sample is below a predetermined threshold, testing the subject for at least one indicator of preeclampsia, and wherein the at least one indicator of preeclampsia is positive, treating the subject for preeclampsia.

According to further aspects of the invention there is provided a method of treating a pregnant female human subject for preeclampsia, the method comprising detecting the level of CD44 and a level of FKBPL in a biological sample of the subject using an agent binding

CD44 and an agent binding FKBPL, wherein the biological sample is of gestational week later than week 30, and wherein a ratio of CD44/FKBPL levels in the biological sample is below a predetermined threshold, testing the subject for at least one indicator of preeclampsia, and wherein the at least one indicator of preeclampsia is positive, treating the subject for preeclampsia.

According to a yet a further aspect of the invention there is provided a method of treating a pregnant female human subject for preeclampsia, the method comprising detecting the level of CD44 and a level of FKBPL in a first biological sample of the subject using an agent binding CD44 and an agent binding FKBPL, wherein the first biological sample is of gestational week 15-30, detecting the level of CD44 and a level of FKBPL in a second biological sample of the subject using an agent binding CD44 and an agent binding FKBPL, wherein the second biological sample is of gestational week later than gestational week 30, and wherein a ratio of CD44/FKBPL levels in the first biological sample is above a first predetermined threshold and a ratio of the CD44/FKBPL levels in the second biological sample is below a second predetermined threshold, testing the subject for at least one indicator of preeclampsia, and wherein the at least one indicator of preeclampsia is positive, treating the subject for preeclampsia.

Thus, according to a further aspect of the invention there is provided a method of treating a pregnant female human subject for preeclampsia, the method comprises detecting the level of CD44 in a first biological sample of the subject using an agent binding CD44, wherein the first biological sample is of gestational week 15-30, detecting the level of CD44 in a second biological sample of the subject using an agent binding CD44, wherein the second biological sample is of gestational week later than gestational week 30, and wherein the CD44 levels in the first biological sample is above a first predetermined threshold and the CD44 levels in the second biological sample is below a second predetermined threshold, testing the subject for at least one indicator of preeclampsia, and wherein the at least one indicator of preeclampsia is positive, treating the subject for preeclampsia.

Thus, according to a further aspect of the invention there is provided a method of treating a pregnant female human subject for preeclampsia, the method comprising detecting the level of CD44 and a level of FKBPL in a first biological sample of the subject using an agent binding CD44 and an agent binding FKBPL, wherein the first biological sample is of gestational week 15-30, detecting the level of CD44 and a level of FKBPL in a second biological sample of the subject using an agent binding CD44 and an agent binding FKBPL, wherein the second biological sample is of gestational week later than gestational week 30, and wherein a ratio of CD44/FKBPL levels in the first biological sample is above a first predetermined threshold and a ratio of the CD44/FKBPL levels in the second biological sample is below a second predetermined threshold, testing the subject for at least one indicator of preeclampsia, and wherein the at least one indicator of preeclampsia is positive, treating the subject for preeclampsia. As mentioned, in some embodiments, the at least one indicator of preeclampsia can include MABP. In other, specific embodiments, MABP is evaluated along with CD44 and CD44/FKBPL, and treatment for preeclampsia is indicated wherein the MABP is elevated, CD44 levels and/or CD44/FKBPL are elevated above a predetermined threshold and the at least one indicator of preeclampsia, which is not MABP, is positive.

It will be noted that, most typically, but not exclusively, in the methods in which the biological sample is of gestational week later than week 30, the predetermined threshold is referenced from values of normal, healthy pregnancies corresponding to gestational week later than gestational week 30. Likewise, typically, but not exclusively, in the methods in which the first biological sample is of gestational week 15-30, and where the second sample is of gestational week later than week 30, the first predetermined threshold is referenced from values of normal healthy pregnancies corresponding to gestational week 15-30 and the second predetermined threshold is referenced from values of normal, healthy pregnancies corresponding to gestational week later than gestational week 30. In specific embodiments, the thresholds (e.g., first and second predetermined thresholds) are determined from values of normal, healthy pregnancies matched with the subject for gestational age and, optionally, additional clinical criteria, as detailed herein.

According to one aspect of the present invention, determining the risk for developing preeclampsia in the pregnant female subject comprises detecting the level of CD44 in a biological sample of the subject.

As used herein, the term “CD44” (HCAM, Hermes antigen, lymphocyte homing receptor LHR, Pgp 1, ECM-III, HUTCH-1) refers to the cell-surface glycoprotein encoded (in humans) by the CD44 gene. In specific embodiments, CD44 is the polypeptide of CD44, GenBank Accession number XP_005253288.1, comprising the amino acid sequence of SEQ ID NO: 4. In other embodiments, the CD44 polypeptide comprises natural variants, fragments and/or polypeptides homologous to the amino acid sequence of CD44. Nucleic acids encoding the human CD44 polypeptide include but are not limited to SEQ ID NO: 3. In specific embodiments, the method comprises detecting a natural variant, fragment and/or homologous polypeptide of CD44 having at least 50%, at least 55%, at least 60%, at least 65%, at least 70%, at least 75%, at least 80%, at least 85%, at least 90%, at least 95% or greater amino acid sequence identity to SEQ ID NO: 22. In other specific embodiments, the CD44 polypeptide, natural variant, fragment and/or homologous polypeptide detected is antigenically cross-reactive with agents binding native CD44 polypeptide. In some embodiments, the CD44 polypeptide detected is cross-reactive with a specific anti-human CD44 antibody, as determined by kinetic assays, saturation assays and/or competitive/modulation binding assays, having similar affinity constant (K) or dissociation constant (Kd) as native human CD44 for binding the anti-human CD44 antibody.

According to another aspect of the present invention, determining the risk for developing preeclampsia in the pregnant female subject comprises detecting the level of CD44 and FK506-binding protein like (FKBPL) in a biological sample of the subject and determining the CD44/FKBPL ratio. As used herein, the term “FKBPL” (DIR1, NG7, WISP39, FKBP prolyl isomerase like) refers to the immunophilin-like protein encoded (in humans) by the FKBPL gene. In specific embodiments, FKBPL is the polypeptide of FKBPL, GenBank Accession number NP_079313, comprising the amino acid sequence of SEQ ID NO: 2. In other embodiments, the FKBPL polypeptide comprises natural variants, fragments and/or polypeptides homologous to the amino acid sequence of FKBPL. Nucleic acids encoding the human FKBPL polypeptide include, but are not limited to, SEQ ID NO: 1.

In specific embodiments, the method comprises detecting a natural variant, fragment and/or homologous polypeptide of FKBPL having at least 50%, at least 55%, at least 60%, at least 65%, at least 70%, at least 75%, at least 80%, at least 85%, at least 90%, at least 95% or greater amino acid sequence identity to SEQ ID NO: 2. In other specific embodiments, the FKBPL polypeptide, natural variant, fragment and/or homologous polypeptide detected is antigenically cross-reactive with agents binding native FKBPL polypeptide. In some embodiments, the FKBPL polypeptide detected is cross-reactive with a specific anti-human FKBPL antibody, as determined by kinetic assays, saturation assays and/or competitive/modulation binding assays, having similar affinity constant (K) or dissociation constant (Kd) as native human FKBPL for binding the anti-human FKBPL antibody.

One example of a binding agent suitable for use in the present invention is an affinity binding molecule specifically binding CD44 or an affinity binding molecule specifically binding FKBPL. In some embodiments, the affinity binding molecule specifically binds at least one epitope of its target (i.e. CD44 or FKBPL polypeptide).

As used herein an “affinity binding molecule” refers to a molecule which binds to a specific antigen.

It should be noted that the affinity can be quantified using known methods such as, Surface Plasmon Resonance (SPR) (described in Scarano S, Mascini M, Turner A P, Minunni M. Surface plasmon resonance imaging for affinity-based biosensors. Biosens Bioelectron. 2010, 25: 957-66) using e.g., a captured or immobilized monoclonal antibody (MAb) format to minimize contribution of avidity, and can be calculated using, e.g., a dissociation constant, Kd, such that a lower Kd reflects a higher affinity.

As used herein the term “K_{D}” refers to the equilibrium dissociation constant between the antigen binding domain and its respective antigen.

According to a specific embodiment, the affinity binding molecule is an antibody or an antibody fragment.

Anti-CD44 antibodies are commercially available to one of skill in the art. Anti-CD44 antibodies suitable for use with the methods and compositions of the invention include, but are not limited to Abcam ab189524, ab51037, ab46793, ab216647, ab16728, ab6124, ab25064 (monoclonal Abs), Abcam ab157104 (polyclonal Ab); Origene UM500099 (monoclonal Ab); R&D Systems, Inc, BBA10; ThemoFisher 14-0441-82, MA5-13890, MA4405 (monoclonal Abs) and PA5-21419. Conjugated (labelled) anti-CD44 antobodies are also widely commercially available. In specific embodiments, CD44 is measured using an ELISA CD44 kit (e.g., ab45912 from Abcam, Cambridge, Mass.).

Anti-FKBPL antibodies are commercially available to one of skill in the art. Anti-FKBPL antibodies suitable for use with the methods and compositions of the invention include, but are not limited to, Abcam ab233426 (polyclonal Ab), ab233409 (monoclonal Ab); Origene TA502051 (monoclonal Ab); ThemoFisher-Invitrogen MA5-25356, MA5-23325 (monoclonal Abs) and PA5-60378, PA5-99231, PA5-66308, PA5-39168 (polyclonal Ab); Proteintech, USA 10060-1-AP and Atlas antibodies HPA043478 (polyclonal Ab). In specific embodiments, FKBPL is measured using an ELISA FKBPL kit (e.g., SEL523Hu from Cloud-Clone Corp, Katy, Tex.). Conjugated (labelled) anti-FKBPL antobodies are also widely commercially available.

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three-dimensional structural characteristics, as well as specific charge characteristics.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR L1 or L1; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of immunological Interest, 5th ed., Public Health Service, NIH, Washington D.C.), location of the structural loop regions as defined by Chothia et al. (see, e.g., Chothia et al., Nature 342:87T883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

a. Fv, defined as a genetically engineered fragment consisting of the variable region of the light chain (VL) and the variable region of the heavy chain (VH) expressed as two chains;

b. single chain Fv (“scFv”), a genetically engineered single chain molecule including the variable region of the light chain and the variable region of the heavy chain, linked by a suitable polypeptide linker as a genetically fused single chain molecule.

c. disulfide-stabilized Fv (“dsFv”), a genetically engineered antibody including the variable region of the light chain and the variable region of the heavy chain, linked by a genetically engineered disulfide bond.

d. Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

e. Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

f. F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);

g. Single domain antibodies or nanobodies are composed of a single VH or VL domains which exhibit sufficient affinity to the antigen; and

h. Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for generating antibodies employ induction of in-vivo production of antibody molecules, screening of immunoglobulin libraries (Orlandi D. R. et al., 1989. Proc. Natl. Acad. Sci. U. S. A. 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U. S. A. 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumine (BSA)] carriers (see, for example, U.S. Pat. Nos. 5,189,178 and 5,239,078]. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. The Fv fragments can comprise VH and VL chains connected by a peptide linker. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

As mentioned, the antibody fragment may comprise a Fc region of an antibody termed “Fcab”. Such antibody fragments typically comprise the CH₂-CH₃ domains of an antibody. Fcabs are engineering to comprise at least one modification in a structural loop region of the antibody, i.e. in a CH₃ region of the heavy chain. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the

FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art; see Winter and co-workers [Jones et al., Nature, 321:522-525 (1986); Riechmann et al., Nature 332:323-327 (1988); Verhoeyen et al., Science, 239:1534-1536 (1988)], (U.S. Pat. No. 4,816,567). In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)], or as described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

In some embodiments, levels of CD44 or FKBPL polypeptide are determined in the biological sample using a molecule comprising an antibody of the invention being conjugated to a functional moiety (also referred to as an “immunoconjugate”) such as a detectable or a therapeutic moiety. The immunoconjugate molecule can be an isolated molecule such as a soluble and/or a synthetic molecule.

Various types of detectable or reporter moieties may be conjugated to the antibody of the invention. These include, but not are limited to, a radioactive isotope (such as ^{[125]}iodine), a phosphorescent chemical, a chemiluminescent chemical, a fluorescent chemical (fluorophore), an enzyme, a fluorescent polypeptide, an affinity tag, and molecules (contrast agents) detectable by Positron Emission Tomagraphy (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Fluorescence detection methods which can be used to detect the antibody when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the anti-CD44 or anti-FKBPL antibody [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

Various methods, widely practiced in the art, may be employed to attach a streptavidin or biotin molecule to the antibody of the invention. For example, a biotin molecule may be attached to the antibody of the invention via the recognition sequence of a biotin protein ligase (e.g., BirA) as described in the Examples section which follows and in Denkberg, G. et al., 2000. Eur. J. Immunol. 30:3522-3532. Alternatively, a streptavidin molecule may be attached to an antibody fragment, such as a single chain Fv, essentially as described in Cloutier SM. et al., 2000. Molecular Immunology 37:1067-1077; Dubel S. et al., 1995. J Immunol Methods 178:201; Huston J S. et al., 1991. Methods in Enzymology 203:46; Kipriyanov S M. et aL, 1995. Hum Antibodies Hybridomas 6:93; Kipriyanov S M. et al., 1996. Protein Engineering 9:203; Pearce L A. et al., 1997. Biochem Molec Biol Intl 42:1179-1188).

Functional moieties, such as fluorophores, conjugated to streptavidin are commercially available from essentially all major suppliers of immunofluorescence flow cytometry reagents (for example, Pharmingen or Becton-Dickinson).

According to some embodiments of the invention, biotin conjugated antibodies are bound to a streptavidin molecule to form a multivalent composition (e.g., a dimmer or tetramer form of the antibody).

TABLE 1
provides non-limiting examples of identifiable moieties which can be
conjugated to the antibody of the invention.

| Identifiable | Amino Acid sequence | Nucleic Acid sequence |
|---|---|---|
| Moiety | (GenBank Acc No.) | (GenBank Acc No.) |
| Green Fluorescent | AAL33912 | AF435427 |
| protein | | |
| Alkaline | AAK73766 | AY042185 |
| phosphatase | | |
| Peroxidase | CAA00083 | A00740 |
| Histidine tag | Amino acids 264-269 | Nucleotides 790-807 |
| | GenBank Accession No. | of GenBank Accession |
| | AAK09208 | No. AF329457 |
| Myc tag | Amino acids 273-283 of | Nucleotides 817-849 |
| | GenBank Accession No. | of GenBank Accession |
| | AAK09208 | No. AF329457 |
| Biotin lygase tag | LHHILDAQKMVWNHR* | |
| orange fluorescent | AAL33917 | AF435432 |
| protein | | |
| Beta galactosidase | ACH42114 | EU626139 |
| Streptavidin | AAM49066 | AF283893 |

*SEQ ID NO: 5

In some aspects of the invention, the method comprises detecting CD44 and/or FKBPL in a biological sample of the subject.

The term “biological sample” encompasses a variety of sample types obtained from an organism and can be used in a diagnostic, prognostic, or monitoring assay. The term encompasses blood and other liquid samples of biological origin or cells derived therefrom and the progeny thereof. The term encompasses samples that have been manipulated in any way after their procurement, such as by treatment with reagents, solubilization, or enrichment for certain components. The term encompasses a clinical sample, and also includes cell supernatants, cell lysates, serum, plasma, biological fluids, and tissue samples. Clinical samples for use in the methods of the invention may be obtained from a variety of sources, particularly blood samples.

Sample sources of particular interest include blood samples or preparations thereof, e.g., whole blood, or serum or plasma, and urine. A sample volume of blood, serum, or urine between about 2 μl to about 2,000 μl is typically sufficient for determining the level of CD44 and/or FKBPL. Generally, the sample volume will range from about 10 μl to about 1,750 μl, from about 20 μl to about 1,500 μl, from about 40 μl to about 1,250 μl, from about 60 μl to about 1,000 μl, from about 100 μl to about 900 μl, from about 200 μl to about 800 μl, from about 400 μl to about 600 μI. In specific embodiments, a suitable initial source for the human sample is a blood sample. As such, the sample employed in the subject assays is generally a blood-derived sample. The blood derived sample may be derived from whole blood or a fraction thereof, e.g., serum, plasma, etc., where in some embodiments the sample is derived from blood, allowed to clot, and the serum or plasma separated and collected to be used to assay. In other embodiments, the sample is derived from blood collected without clotting (e.g., along with anti-coagulant such as EDTA, citrate, heparin) and then serum or plasma collected for assay.

In some embodiments the sample is a serum or serum-derived sample. Any convenient methodology for producing a fluid serum sample may be employed. In many embodiments, the method employs drawing venous blood by skin puncture (e.g., finger stick, venipuncture) into a clotting or serum separator tube, allowing the blood to clot, and centrifuging the serum away from the clotted blood. In some embodiments, the blood is collected without clotting (e.g., collected into separator tubes coated with anti-coagulant).

The serum is then collected and stored until assayed. Once the patient derived sample is obtained, the sample can then be assayed to determine the level of CD44 and/or FKBPL.

In some embodiments, the subject sample may be treated in a variety of ways so as to enhance detection of the one or more of CD44 and FKBPL. For example, where the sample is blood, the red blood cells may be removed from the sample (e.g., by centrifugation) prior to assaying. Such a treatment may serve to reduce the non-specific background levels of detecting the level of CD44 or FKBPL using an affinity reagent. Detection of CD44 and/or FKBPL may also be enhanced by concentrating the sample using procedures well known in the art (e.g. acid precipitation, alcohol precipitation, salt precipitation, hydrophobic precipitation, filtration (using a filter which is capable of retaining large polypeptides), affinity purification). In some embodiments, the pH of the test and control samples will be adjusted to, and maintained at, a pH which approximates neutrality (i.e. pH 6.5-8.0). Such a pH adjustment will prevent complex formation, thereby providing a more accurate quantitation of the level of marker in the sample. In embodiments where the sample is urine, the pH of the sample is adjusted, and the sample is concentrated in order to enhance the detection of the marker.

The subject sample is typically obtained from the individual during the second or third trimester of gestation. By “gestation” it is meant the duration of pregnancy in a mammal, i.e. the time interval of development from fertilization until birth, plus two weeks, i.e. to the first day of the last menstrual period. By the second or third trimester, it is meant the second or third portions of gestation, each segment being 3 months long (in a human subject). Thus, for example, by the “first trimester” is meant from the first day of the last menstrual period through the 13th week of gestation; by the “second trimester” it is meant from the 14th through 27th week of gestation; and by the “third trimester” it is meant from the 28th week through birth, i.e. 38-42 weeks of gestation. Put another way, a subject sample may be obtained at about weeks 14 through 42 of gestation, at about weeks 18 through 42 of gestation, at about weeks 20 through 42 of gestation, at about weeks 24 through 42 of gestation, at about weeks 30 through 42 of gestation, at about weeks 34 through 42 of gestation, at about weeks 38 through 42 of gestation.

Thus, in some embodiments, the subject sample may be obtained early in gestation, e.g. between weeks 14-30 of gestation, e.g. at week 14, 15,16, 17, 18, 19, 20, 21, 22, 23, 24, 25, 26, 27, 28, 29 or 30 of gestation, or at weeks 20 or more, or weeks 15-30 of gestation. In other embodiments, the subject sample may be obtained later in gestation, for example, at or after 30 weeks of gestation, e.g., at week 31, 32, 33, 34, 35, 36, 37, 38, 39, 40, or week 41 of gestation.

The inventors have shown that CD44 levels can be an effective predictor of risk for developing preeclampsia in early pregnancy. Thus, in specific embodiments, the method of the invention comprises detecting CD44 levels in samples from gestational week 20 and on. In some embodiments, CD44 levels are detected at gestational weeks 20-30, and more specifically at week 20, 21, 22, 23, 24, 25, 26, 27, 28, 29 or gestation week 30. It will be appreciated that “gestational week” refers to any time during the indicated gestational week, as defined hereinabove.

The inventors have also shown that the CD44/FKBPL ratio can also be an effective predictor of risk for developing preeclampsia in early pregnancy. Thus, in specific embodiments, the method of the invention comprises detecting CD44 and FKBPL levels and determining the CD44/FKBPL ratio in samples from gestational week 15 and on. In some embodiments, CD44 and FKBPL ratios are determined at gestational weeks 15-30, and more specifically at week 15, 16, 17, 18, 19, 20, 21, 22, 23, 24, 25, 26, 27, 28, 29 or gestation week 30.

As used herein, the term “managing pregnancy” refers to prescribing or providing any means, treatments, medications, monitoring or other methods for maintenance of the pregnancy and the health and well-being of the fetus and/or the mother. As used herein, the phrase “high risk pregnancy management” refers to close monitoring of clinical and other indicators of preeclampsia and/or fetal distress, and suitable interventions to allow maintenance of healthy fetal development towards term. In specific embodiments, indicators of preeclampsia include, but are not limited to hypertension, proteinuria, low platelet count, impaired liver function, signs of kidney problems (other than proteinuria), pulmonary edema and headaches or visual disturbances.

In some embodiments, “hypertension” in the context of preeclampsia is stage 1 hypertension, defined as systolic pressure between 130-139 mm Hg or diastolic pressure between 80-89 mm Hg. In other embodiments, “hypertension” is stage 2 hypertension, defined as systolic pressure at least 140 mm Hg or diastolic pressure at least 90 mm Hg. In specific embodiments, “normotension” is defined as MABP equal to or lower than 85 mm Hg, and “hypertension” is defined as MABP greater than 85 mm Hg.

In some embodiments, “proteinuria” refers to 30 or more mg/dL protein in a urine sample (e.g., +1 or greater on a Uniplus urinalysis strip). Greater than 100 mg/dL (+2 on a Uniplus strip) may indicate severe proteinuria. In other embodiments, proteinuria is measured in a 24-hour urine collection, wherein proteinuria is indicated by at least 300 mg protein/24 hours.

In some embodiments, “low platelet count”, or thrombocytopenia refers to a drop in platelet counts, as measured in a blood test (CBC or complete blood count). Normal platelet count is between 150-400 million/mL. A drop in platelet count to between 100 and 150 million platelets/ml blood is considered mild gestational thrombocytopenia, and a platelet count below 70 million/mL is considered severe gestational thrombocytopenia. In some embodiments the subject's platelet count is between 100-150 million /mL. In other embodiments, platelet count is below 100 million/mL.

As used herein, “liver function” refers to a panel of markers indicating the state of a patient's liver. Liver function tests (also known as a “hepatic panel”) include, but are not limited to measuring total bilirubin, alanine transaminase (ALT), aspartate transaminase (AST), the AST/ALT ratio, gamma glutamyl transpeptidase (GGT) and albumin measured in samples of the patient's blood or blood fractions. In some embodiments additional tests include determining coagulation factors and function (e.g., prothrombin time (PT), international normalized ratio (INR), PT/INR), 5′ nucleotidase (5NTD), ceruloplasmin, alpha-fetoprotein (AFP), serum glucose and lactate dehydrogenase (LDH) are performed to evaluate liver function impairment in pregnancy.

As used herein, “kidney function (or renal function) (other than proteinuria)” refers to a panel of markers and functional parameters indicating the state of a patient's kidney and kidney physiology. In some embodiments, kidney function markers include plasma uric acid (upper limits of normal are 5-5.5 mg/dL), blood urea nitrogen (BUN) (normal values in pregnancy are around 9 mg/dL, and values above 14 mg/dL are indicative of renal dysfunction) and plasma creatinine (normal creatinine in pregnancy is around 0.5 mg/dL, and values exceeding 0.8 mg/dL are indicative of renal dysfunction). It will be noted that tests (e.g. markers of renal function) in pregnancy should be interpreted in relation to changes in other parameters of kidney function, including but not limited to changes in plasma volume, glomerular filtration and tubular reabsorption, that normally occur with advancing gestation.

In some embodiments the at least one indicator of preeclampsia is pulmonary edema. “Pulmonary edema” is diagnosed on the basis of a history of dyspnea, pulmonary rales, hypoxemia (PaO2 less than 80 mmHg or SatO2 less than 90% with room air) and/or air-space opacities on chest radiograph.

In some embodiments the at least one indicator of preeclampsia is visual disturbances”. As used herein, “visual disturbances” refers to visual symptoms or phenomena associated with preeclampsia, such as but not limited to blurry vision, diplopia, amaurosis fugax (temporary loss of vision), photopsia, scotoma and homonymous hemianopsia. In other embodiments the at least one indicator of preeclampsia is “headaches”. The term “headache” refers to head pain, frequently characterized as throbbing, squeezing, constant, unrelenting, or intermittent head pain. The location may be in one part of the face or skull or may be generalized involving the whole head.

As used herein, the term “treating” a preeclampsia refers to prescribing or providing any treatment of a preeclampsia in a female mammal and includes: (a) preventing the preeclampsia from occurring in a subject which may be predisposed to preeclampsia (high risk) but has not yet been diagnosed as having it; (b) inhibiting a recognized preeclampsia, i.e., arresting its development; or (c) relieving the preeclampsia, i.e., causing regression of the preeclampsia.

“Preventing preeclampsia” may include, but is not limited to prescription of statins (e.g. pravastatin), fibrates (e.g. fenofibrate), stilbenoids (e.g. resveratrol) or metformin.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to the pregnant subject and/or fetus). The dosage, schedule and duration of treatment can vary, depending on the severity of pathology and the selected type of treatment, and those of skills in the art are capable of adjusting the type of treatment with the dosage, schedule and duration of treatment. Preeclampsia treatments known in the art suitable for use with the present invention may include bed rest, drinking extra water, a low salt diet, anti-hypertensive treatment (e.g., labetolol, nifedipine, methyldopa and the like), corticosteroids, in severe situations, seizure prophylaxis (e.g., magnesium sulfate), inducing pregnancy, and the like. In some embodiments, treatment of preeclampsia comprises administration of a FKBPL agonist. In specific embodiments, the FKBPL agonist is prescribed for subjects having high risk of preeclampsia before gestational week 30, and/or reduced blood FKBPL or elevated CD44/FKBPL ratio.

As mentioned hereinabove, the methods of the present invention can be used to determine treatment strategy and regimen for pregnant females at risk of developing preeclampsia or suffering from one or more symptoms of preeclampsia. Thus, in some embodiments, there is provided an agent for treatment of preeclampsia for use in a pregnant human female subject exhibiting a level of CD44 greater than a predetermined threshold in a biological sample therefrom, wherein the sample is from 20-30 weeks gestation; or lower than a second predetermined threshold, wherein the sample is from 30 or more weeks gestation.

Also provided is an agent for treatment of preeclampsia for use in a pregnant human female subject exhibiting a ratio of CD44/FKBPL greater than a predetermined threshold in a biological sample therefrom, wherein the sample is from 15-30 weeks gestation, or lower than a second predetermined threshold wherein the sample is from 30 or more weeks gestation. As mentioned hereinabove, in some embodiments, the pregnant human female subject further exhibits an elevated MABP.

In some embodiments, the agent for treatment of preeclampsia comprises a drug selected from the group consisting of an anti-hypertensive drug, a corticosteroid and an anticonvulsive drug.

Also provided are reagents, systems and kits thereof for practicing one or more of the above-described methods. The subject reagents, systems and kits thereof may vary greatly. Reagents of interest include reagents specifically designed for use in producing the above-described marker level representations of preeclampsia markers from a sample, for example, one or more detection elements, e.g., antibodies or peptides for the detection of CD44 and/or FKBPL protein, oligonucleotides for the detection of nucleic acids, etc. In some instances, the kit comprises a first agent which specifically binds soluble CD44, a second agent which specifically binds soluble FKBPL, and normal reference samples for CD44 and FKBPL. In specific embodiments, the normal reference sample for CD44 and reference sample for FKBPL is a sample containing a level of CD44 or FKBPL, respectively, found in a pregnant human female subject not having preeclampsia or risk to develop preeclampsia, early-onset preeclampsia or eclampsia. In other embodiments, the first and/or second agents may detectably labeled or coupled to an enzyme, e.g. a fluorescent labeled, radioactive labeled or immunoconjugated antibody, as detailed hereinabove. The agents binding CD44 and/or FKBPL can be an antibody, or CD44 or FKBPL-binding fragment thereof. In some embodiments, the antibody is a monoclonal antibody. In other embodiments, the agent binding CD44 and/or the agent binding FKBPL are immobilized on a solid surface, e.g., bound to beads (microspheres), ELISA plate, etc, as detailed hereinabove.

In some embodiments, the system or kit comprises a test strip (e.g., lateral flow test strip) also known as dipstick, preferably, though not necessarily, encased in a housing, designed to be read by the subject or medical professional, and in some embodiments, the assay performed with the test strip is a sandwich immunoassay. Such devices are impregnated with reagents that specifically indicate the presence of a given molecule, e.g., CD44 and/or FKBPL, by changing colour or other indication, typically visual, upon contact with a sample. Types of subject's samples have already been defined herein. In some embodiments, an antibody is labelled by conjugation to a physically detectable label, and upon contacting with a sample containing CD44 and/or FKBPL forms a complex. The antibody-CD44 and/or FKBPL complex is then contacted with a second antibody, which recognizes the complex and which is immobilized on a solid support within the device. The second antibody captures the antibody-CD44 complex or antibody-FKBPL complex to form an antibody-CD44 and/or FKBPL-antibody sandwich complex, and the resulting complex, which is immobilized on the solid support, is detectable by virtue of the label. A test strip may then be inserted into a reader, where a signal from the label in the complex is measured. Alternatively, a test strip could be inserted into the reader prior to addition of the sample. Alternatively, the presence of CD44 and/or FKBPL is visualised by a subject as a change of colour of at least a part of a device. Dipsticks are usually made of paper or cardboard. Usually, additional molecules are present in a device as a positive or negative control. A typical positive control could be an antibody recognizing a molecule which is known to be present in a sample to be tested. A typical negative control could be an antibody recognizing a molecule which is known to be absent in a sample to be tested.

In some embodiments, the normal reference sample of CD44 contains CD44 in a range of concentrations between 20 and 300 ng/ml, between 25 and 275 ng/ml, 35 and 250, 50 and 225, 75 and 200, 100 and 180, 110 and 160, 100 and 200 ng/ml, and the normal reference sample of FKBPL contains FKBPL in a range of concentrations between 0.1 and 5 ng/ml, between 0.15 and 4.75 ng/ml, 0.2 and 4.50, 0.5 and 4.2, 0.75 and 4.0, 1.0 and 3.80, 1.5 and 3.5, 1.0 and 5.0 ng/ml.

Another type of such agent is an array of probes, collections of primers, or collections of antibodies that include probes, primers or antibodies (also called reagents) that are specific for CD44 and FKBPL. Such an array may include reagents specific for additional genes/proteins/cofactors that are not listed above, such as probes, primers, or antibodies specific for genes/proteins/cofactors whose expression pattern are known in the art to be associated with preeclampsia, e.g. and sFlt-1 (VEGF-RI) and PIGF.

In some embodiments, there is provided a composition of matter comprising a biological sample of a pregnant female human subject, an agent binding CD44 and a second agent binding FKBPL. The agent binding CD44 and the second agent binding FKBPL can be any one of the agents described in detail hereinabove, e.g. specific antibodies to CD44 and to FKBPL, respectively, and the biological sample of the pregnant female may be any one of the samples described hereinabove. In specific embodiments, the sample is a blood fraction (plasma or serum, whole blood) for example, from 15 weeks on, from a pregnant female in mid-gestation (week 15-30). In other embodiments, the sample is a blood fraction from a pregnant female in late gestation, e.g., at later than 30 weeks gestation. In further examples, the composition of matter comprises an (a) first portion of a biological sample of a pregnant female human subject comprising the agent binding CD44, and (b) a second portion of the biological sample of the pregnant female human subject comprising the agent binding FKBPL.

In some instances, a system may be provided. As used herein, the term “system” refers to a collection of reagents, however compiled, e.g., by purchasing the collection of reagents from the same or different sources. In some instances, a kit may be provided. As used herein, the term “kit” refers to a collection of reagents provided, e.g., sold, together. For example, the antibody-based detection of the sample proteins, respectively, may be coupled with an electrochemical biosensor platform that will allow multiplex determination of CD44 and FKBPL for personalized preeclampsia care. The systems and kits of the subject invention may include the above-described arrays, gene-specific primer collections, or protein-specific antibody collections, as well as one or more additional reagents employed in the various methods, which may be either premixed or separate. The subject systems and kits may also include one or more preeclampsia phenotype determination elements, e.g. a reference or control sample or marker representation that can be employed, e.g., by a suitable experimental or computing means, to make a preeclampsia prognosis based on an “input” marker level profile.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

Reference is now made to the following examples, which together with the above descriptions, illustrate the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Enzyme Linked Immunosorbent Assay (ELISA) of Pregnant Human Plasma Samples

Plasma samples from a cohort of nulliparous women with singleton pregnancies were obtained at week 15 and 20 of gestation before the diagnosis of preeclampsia. Plasma FKBPL (Cloud-Clone Corp., China) and CD44 (Abcam, UK) levels were measured at 15 and 20 weeks' gestation in women who subsequently developed preeclampsia post 20 weeks' gestation, and the levels were compared to age and BMI matched healthy controls.

The average intra- and inter-assay CV for FKBPL was 5.5% and 17.1% respectively. Each ELISA plate included a randomised selection of preeclampsia and control samples at each time point in gestation. The operators were also blinded. With respect to the CD44 ELISA, the average inter-assay CV was 6.9% and the average intra-assay CV % was 3.9%. Any sample with a CV (%) over 15% was reanalysed and samples were only included if the CV % was less than 15%. Two-tailed t-test or Mann-Whitney statistical analyses were performed depending upon data distribution.

Plasma FKBPL and CD44 was also measured in samples from a separate cohort of pregnant women after diagnosis of preeclampsia (n=18), and matched, normotensive pregnant controls (n=14).

EDTA plasma samples obtained from whole blood were centrifuged for 15 minutes at 2,000 rpm at 4° C. Upon separation, plasma was stored at −80° C. until further usage. Plasma was diluted 2-fold for FKBPL (Cloud-Clone Corp., China) or 40-fold for CD44 (Abcam, UK) using standard diluent provided by the kit.

### Cell Culture

A total of four cell lines (BeWo, Jar, human umbilical vein endothelial cells (HUVECs), bone-marrow derived mesenchymal stem cell (MSCs)) were used in subsequent investigations. All cell lines were authenticated by short tandem repeat (STR) profiling performed by the suppliers and routinely tested for Mycoplasma.
a. Human Placenta Choriocarcinoma (BeWo) were maintained in DMEM/F12 medium supplemented with 10% foetal bovine serum (FBS).
b. Human Placenta Choriocarcinoma (Jar) were maintained in RPMI-1640 medium supplemented with 10% FBS.
c. Human Umbilical Vein Endothelial Cells (HUVECs) were maintained in endothelial cell growth medium MV2, supplemented with low serum growth supplement (LSGS) on gelatin coated tissue culture flasks.
d. MSCs were maintained in αMEM supplemented with 16.5% heat-inactivated foetal bovine serum (FBS), 50 μg/ml penicillin-streptomycin (PS), and 4 mM L-glutamine, and used no later than passage 7.

All cell culture consumables were purchased from Thermo Fisher Scientific (UK) unless otherwise stated. All cells were initially grown in monolayers in vented 75 cm² tissue culture flasks (Nunc; Thermoscientific, Denmark) at 37° C., in an atmosphere of 5% CO₂ and 95% air.

Treatment with MSCs Conditioned Medium

MSCs were seeded in six well plates in required cell culture media for HUVEC, BeWo and Jar at a ratio of 1 MSC to every 5 HUVEC, BeWo or Jar. The cells were allowed to adhere and conditioned in the cell culture media for 24 h under 21% O₂ conditions. Prior to the day of experimentation, the conditioned medium was aspirated off, collected, and centrifuged for 5 minutes at 1,200 rpm to remove cell debris. Conditioned media was then pre-equilibrated to 21% (Normoxia) or 1% (Hypoxia) O₂ prior to being used for other downstream experiments.

### Tubule Formation Assay

To assess the ability of HUVECs to form tube-like structures, 7×10⁵ HUVEC were re-suspended in 500 μl MSC-CM, or control media, and stained with calcein (2 μg/ml; Thermo Fisher Scientific, UK) prior to being seeded on phenol red free reduced-growth factor Matrigel under 21% and 1% O₂ for 6 h. Tubule formation was imaged by randomly capturing 6 images per well using a DM18 inverted florescence microscope. Total tube length was quantified using Image J software (NIH, USA).

### Wound Scratch Assay

BeWo, Jar and HUVECs were allowed to reach confluence at 37° C. in 5% CO₂. A single vertical ‘scratch’ wound was made using a sterile P200 tip from the top to bottom of each well, running through the horizontal line. Cells were washed twice with PBS to remove cell debris and 500 μl serum free cell growth medium was added to each well. The wound area of each well just above and just below the horizontal line was imaged using a DMI8 inverted light microscope, along with tile scan co-ordinates available with AxioVision Rel. 4.8 software.

Following baseline measurement, serum free media was replaced with 500 μl MSC conditioned medium, and prescribed cultivation media or serum free media (pre-equilibrated in 21% or 1% O₂). Plates were incubated at 37° C. in 21% or 1% O₂ for 24 h before wound areas were re-imaged. The area of the wound site at baseline and at 24 h was measured using ImageJ v1.48. The values obtained were used to calculate the percentage of wound closure over 24 h.

### Real Time-Polymerase Chain Reaction

RNA was extracted using TRIzol™ reagent (Thermo Fisher Scientific, United Kingdom) and real-time qPCR performed as described previously. ²² Following RNA extraction, reverse transcription using custom made master mix (Thermo Fisher Scientific,

UK) was carried out. cDNA was prepared for real-time qPCR using the Roche Lightcycler Probes 480 Master kit (Roche, USA) and Roche Realtime Ready TaqMan gene expression mono hydrolysis probes for human FKBPL, CD44 and 18S (Roche, USA). The resulting crossing points (Cp) were calculated using the Roche LightCycler 480 software and quantified using the standard curve efficiency. Sample Cp values were corrected to 18S levels. Data analysis was carried using the ΔΔCT method.

### Western Blotting

Following migration and invasion assays, BeWo, Jar and HUVEC cell lysates were harvested using RIPA buffer (SantaCruz Biotechnology, USA) supplemented with protease and phosphatase inhibitor cocktails (Roche, UK) and subjected to western blotting. Cell lysates were reduced in 4× and 10× Bolt LDS Sample buffer (Thermo Fisher Scientific, United Kingdom) and subjected to Western blotting. Blots were probed with specific primary antibody and appropriate HRP-linked secondary IgG (Cell signalling, UK) at 1:10,000. The membrane was imaged with G:Box (SynGene, India) after incubation using either West Dura or Femto chemiluminescent (Thermo Fisher Scientific, UK). Antibodies CD44H (R&D Systems, cat: BBA10); FKBPL (Proteintech, USA cat: 10060-1-AP); anti-GAPDH (cell signalling, UK, cat: G9545); all antibodies were used at 1:1,000 unless otherwise stated.

### Statistical Analysis

The data was analysed using a two-tailed t-test or Mann-Whitney, or one-way or two-way ANOVA followed by Sidak's or Tukey's multiple comparisons tests where appropriate and adjusted p values reported. Results are expressed as the mean±the standard error of the mean (SEM) and values were considered statistically significant if p<0.05. Analysis was performed using Prism 5 software (Graph Pad Software, La Jolla, Calif., USA).

The statistical analyses of the results in relation to FKBPL, CD44 and the clinical characteristics of the patient cohort, were performed using SPSS 20.0 (IBM, New York, USA) After initial testing for normality (Shapiro-Wilks test), differences between groups (preeclampsia vs. controls) were tested using independent samples t-test or Mann-Whitney, where appropriate. Preeclampsia and control samples were matched for age and BMI. Receiver Operating Characteristic (ROC) analysis was used to obtain cut off values for primary variables (CD44/FKBPL ratio and mean arterial blood pressure (MABP)) measured at 20 weeks gestation. Participants were divided into either a group with low levels (below the cut-off) or a group with high levels (above the cut-off value) of the parameters in question. The contribution of the CD44/FKBPL ratio to the risk of preeclampsia development was assessed using binary and multinominal logistic regression, which included age, body mass index (BMI), weight change and MABP as the possible confounders. Multinomial logistic regression was used to test the combined effect of the CD44/FKBPL ratio and MABP on the risk of developing preeclampsia. Significance was set at p<0.05.

### Results

### Example I: The CD44/FKBPL Ratio is an Effective Parameter for Early Determination of Risk of Preeclampsia

The inventors investigated the early biomarker potential of FKBPL and CD44 individually, and in combination with important clinical parameters, using plasma samples from nulliparous women with singleton pregnancies at 15 and 20 weeks' gestation prospectively followed throughout pregnancy.

Patient characteristics are presented in Table 2.

TABLE 2
Clinical characteristics of pregnant women at 15 and 20 weeks of pregnancy

| | 15 weeks of pregnancy | | | 20 weeks of pregnancy | | |
|---|---|---|---|---|---|---|
| | | | p- | | | p- |
| Variable | Controls | Cases | value | Controls | Cases | value |
| Age (years) | 30.1 ± 3.6 | 30.2 ± 4.2 | 0.95 | 30.1 ± 3.6 | 30.2 ± 4.2 | 0.95 |
| BMI (kg/m²) | 25.8 ± 4.0 | 26.9 ± 5.0 | 0.16 | n/a | n/a | n/a |
| Gestational age at | 38.2 ± 13.3 | 37.9 ± 13.3 | 0.9 | 38.2 ± 13.3 | 37.9 ± 13.3 | 0.9 |
| delivery (weeks) | | | | | | |
| Weight change | n/a | n/a | n/a | 3.2 ± 1.9 | 2.9 ± 2.0 | 0.97 |
| (kg) | | | | | | |
| SBP (mmHg) | 103.2 ± 8.9 | 109.8 ± 10.5 | <0.001 | 107.7 ± 10.3 | 112.1 ± 10.5 | 0.02 |
| DBP (mmHg) | 64.9 ± 6.6 | 69.1 ± 7.1 | <0.001 | 66.3 ± 6.2 | 70.1 ± 7.6 | 0.002 |
| MAP (mmHg) | 77.7 ± 6.8 | 82.6 ± 7.7 | <0.001 | 80.1 ± 7.1 | 84.1 ± 8.0 | 0.003 |
| Random glucose | 5.1 ± 0.9 | 5.5 ± 1.2 | 0.049 | 5.4 ± 1.1 | 5.8 ± 1.0 | 0.047 |
| (mmol/l) | | | | | | |
| CD44 (ng/ml) | 115.9 ± 24.5 | 121.7 ± 28.7 | 0.23 | 115.3 ± 26.4 | 127.5 ± 30.2 | 0.020 |
| FKBPL (ng/ml) | 1.0 | 0.72 | 0.034 | 0.89 | 0.65 | 0.011 |
| | [0.59-1.30] | [0.52-1.10] | | [0.61-1.16] | [0.44-1.08] | |
| CD44/FKBPL | 128.8 | 143.1 | 0.018 | 143.1 | 181.1 | 0.004 |
| ratio | [91.2-176.1] | [113.2-259.2] | | [101.3-176.3] | [121.3-302.4] | |

All values are presented as mean ± SD or median with interquartile range (IQR)
Two-tailed unpaired t-test (normalized distribution) or Man-Whitney (non-normalized distribution)
BMI: body mass index;
SBP: systolic blood pressure;
DBP: diastolic blood pressure;
MAP: mean arterial blood pressure
Cases = preeclampsia

CD44 is a Predictive Biomarker for Preeclampsia from Gestational Week 20:

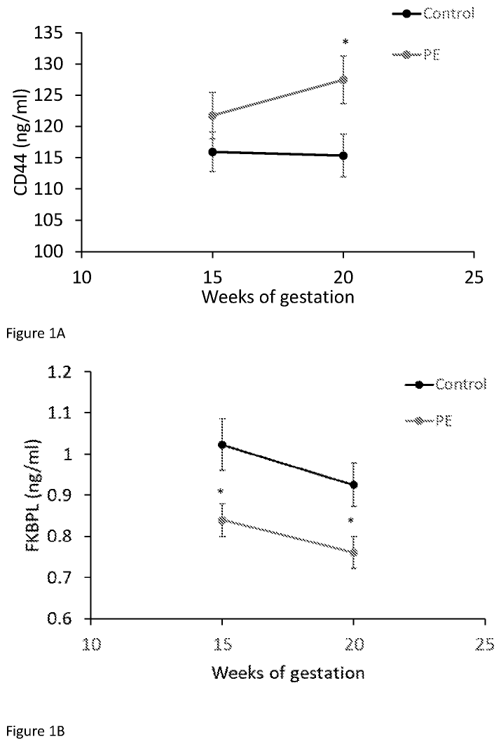

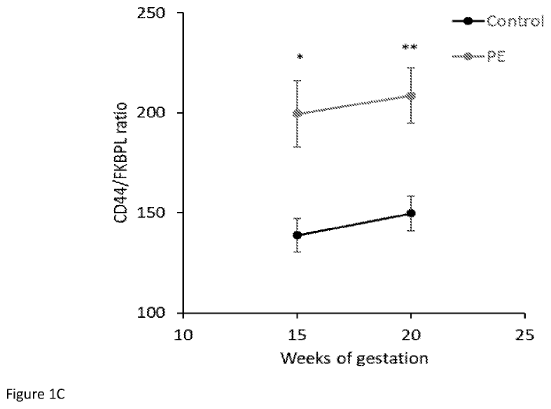

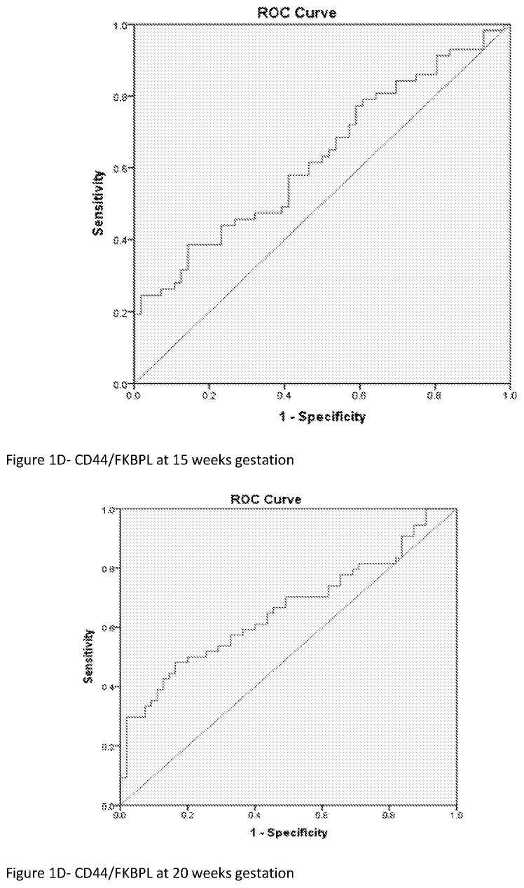

When CD44 was measured in plasma of normal pregnant women and then in women who developed preeclampsia post-20 weeks gestation, no difference in CD44 plasma levels was observed at 15 weeks' gestation between women who later developed preeclampsia (121.7 35 3.7 SEM, n=60) and healthy controls who maintained normal pregnancies (115.9 ng/ml±3.2 SEM, n=60; FIG. 1A, p=0.23).

At 20 weeks' gestation, CD44 plasma levels were higher in the group that later developed preeclampsia compared to controls maintaining normal pregnancies (127.5 ng/ml±3.8 SEM, n=62 vs. 115.3 ng/ml±3.4 SEM, n=60; p=0.02) (FIG. 1A).

FKBPL is a Predictive Biomarker for Preeclampsia from Gestational Week 15:

FKBPL plasma levels were significantly lower in women who later developed preeclampsia (0.84 ng/ml±0.06 SEM, n=61) compared to healthy controls who maintained normal pregnancies (1.02 ng/ml±0.06 SEM, n=59) at 15 weeks' gestation (FIG. 1B, p=0.03).

At 20 weeks' gestation, FKBPL plasma levels (0.76 ng/ml±0.05 SEM, n=57 vs. 0.92 ng/ml±0.05 SEM, n=58;, p=0.01) were lower in the group that later developed preeclampsia compared to controls maintaining normal pregnancies, respectively (FIG. 1B).

### CD44/FKBPL Ratio is an Effective, Early Indicator of the Risk of Preeclampsia

When CD44 and FKBPL were combined as CD44/FKBPL ratio, the differences between the group later developing preeclampsia and control group demonstrated an almost 1.5-fold increase in plasma CD44/FKBPL levels [15^{th} week: 199.5±16.5 SEM, n=57 vs. 138.7±8.3 SEM, n=56 (p=0.02); 20^{th} week: 208.5±13.9, n=54 vs. 149.6 ng/ml±8.7, n=55 (p=0.004) FIG. 1C]. The CD44/FKBPL ratio as a predictive biomarker of preeclampsia, on its own, provided the AUC on the receiver operating characteristic (ROC) curve of 0.629 (95% CI 0.527-0.732; p=0.02) at 15 weeks' gestation and 0.659 (95% CI 0.55-0.76; p=0.004) at 20 weeks' gestation (FIG. 1D).

Based on the ROC analysis, it was possible to determine that a CD44/FKBPL ratio in the range of 100-300 at 20 week's gestation represents a 1.5 to 5.0 fold increased risk for developing preeclampsia later in pregnancy. Specific cut-off point could also be extrapolated from the data: a suitable cut-off point for the CD44/FKBPL ratio of 143.6 (sensitivity=0.7, specificity=0.5) and 155.1 (sensitivity=0.6 and specificity 0.6) for prediction of the risk of preeclampsia at 20 weeks' gestation. Using these cut-off points, univariate logistic regression model demonstrated, that women with a CD44/FKBPL ratio above 143.6 at 20 week's gestation had a 2.5-fold increased risk of developing preeclampsia later in pregnancy (OR=2.5 95% CI 1.12-5.41, p=0.02) and 155.1 cut-off point had a 2.4-fold increased risk of developing preeclampsia later in pregnancy (OR=2.4 95% CI 1.09-5.08, p=0.03).

Multivariate logistic regression models using BMI, age, weight change and mean arterial blood pressure (MABP) as confounders, demonstrated that the CD44/FKBPL ratio was independently associated with preeclampsia (143.6 cut-off: OR=2.3 95% CI 1.03-5.2, p=0.04; 155.1 cut-off: OR=2.3 95% CI 1.05 - 5.2, p=0.04; Table 2 below). The combined effect of CD44/FKBPL and MABP on the risk of developing preeclampsia was investigated using multinomial logistic regression models. Participants were divided into four groups: (Group 1) low-risk (reference) group—women with low levels of both the CD44/FKBPL ratio (<143.6 or 155.1) and MABP (<82.5 mmHg); (Group 2) medium risk 1 group included pregnancies with a high CD44/FKBPL (>143.6 or 155.1) and low MABP (<82.5 mmHg); (Group 3) medium risk 2 group included pregnancies with a low CD44/FKBPL ratio and high MABP, and (Group 2) high-risk group (CD44/FKBPL>143.6 or 155.1 and MABP>82.5 mmHg) (Table 2).

Analysis of the integrated data indicated that combining the CD44/FKBPL ratio with MABP provided increased accuracy for prediction of risk of preeclampsia at 20 week's gestation, effectively extending the range of OR from 1.5-5.0 without MABP to up to 3-12 fold with MABP >82.5. Statistical significance was confined to the high-risk model, which showed a 3.9-fold (cut-off point 143.5: 95% CI 1.3 - 11.8, p=0.02, Table 3a) and a 4.1-fold (cut-off point 155.1: 95% CI 1.4 - 12.4, p=0.01, Table 3b) increased risk of developing preeclampsia.

Tables 3a and 3b: Odds ratio (OR) for developing preeclampsia based on the combination of low or high levels of MABP and low or high levels of the CD44/FKBPL ratio at 20 weeks gestation based on the low risk reference value

TABLE 3a

| MABP and CD44/FKBPL ^{a} | Controls, n(%) | Cases, n(%) | OR (95% CI) | p-value |
|---|---|---|---|---|
| Low risk ^{b} | 16 (29) | 7 (13) | 1.0 | |
| Medium risk 1 ^{c} | 13 (24) | 14 (26) | 2.46 (0.78-7.90) | 0.130 |
| Medium risk 2 ^{d} | 12 (22) | 9 (17) | 1.71 (0.49-5.92) | 0.394 |
| High risk ^{e} | 14 (25) | 24 (44) | 3.92 (1.30-11.84) | 0.016 |

TABLE 3b

| MABP and CD44/FKBPL ^{a} | Controls, n(%) | Cases, n(%) | OR (95% CI) | p-value |
|---|---|---|---|---|
| Low risk ^{b} | 18 (33) | 8 (15) | 1.0 | |
| Medium risk 1 ^{c} | 11 (20) | 13 (24) | 2.66 (0.84-8.46) | 0.098 |
| Medium risk 2 ^{d} | 15 (27) | 13 (24) | 1.95 (0.64-5.95) | 0.241 |
| High risk ^{e} | 11 (20) | 20 (37) | 4.09 (1.35-12.43) | 0.013 |

Taken together, these results indicate that, measured at 20 weeks gestation or later, soluble CD44 is an effective biomarker of the risk of developing preeclampsia at or after 30 weeks gestation. Surprisingly, the results also indicate that, although CD44 is not elevated at 15 weeks gestation, the plasma CD44/FKBPL ratio is an even more effective indicator of the propensity to and/or risk of developing preeclampsia at or after 30 weeks gestation, growing in sensitivity and specificity by 20 weeks gestation. ^{a} Cut off value for CD44/FKBPL ratio (143.5 (Table 3a) or 155.1 (Table 3b)) and MABP (82.5 mmHg) were determined based on the ROC analysis^{b} Low risk: Low CD44/FKBPL and low MABP—reference category^{c} Medium risk 1: High CD44/FKBPL and low MABP^{d} Medium risk 2: Low CD44/FKBPL and high MABP^{e} High risk: High CD44/FKBPL and high MABPOR, odds ratio; CI, confidence interval

Addition of the parameter of MABP further heightens the predictive value of the plasma CD44/FKBPL ratio for early detection of presence or risk of preeclampsia in pregnant women, allowing for greater accuracy in screening for and applying treatment for preeclampsia after 20 weeks gestation.

In summary, high CD44/FKBPL ratio is a risk factor independent of BMI, age, weight change and MABP for preeclampsia. In combination with the MABP, a high CD44/FKBPL ratio and high MABP is associated with an approximately 4-fold increased risk of preeclampsia.

### Example II: Changes in FKBPL and CD44 in Preeclampsia

Plasma FKBPL and CD44 levels and FKBPL from MSCs showed a reverse trend in women diagnosed with preeclampsia.

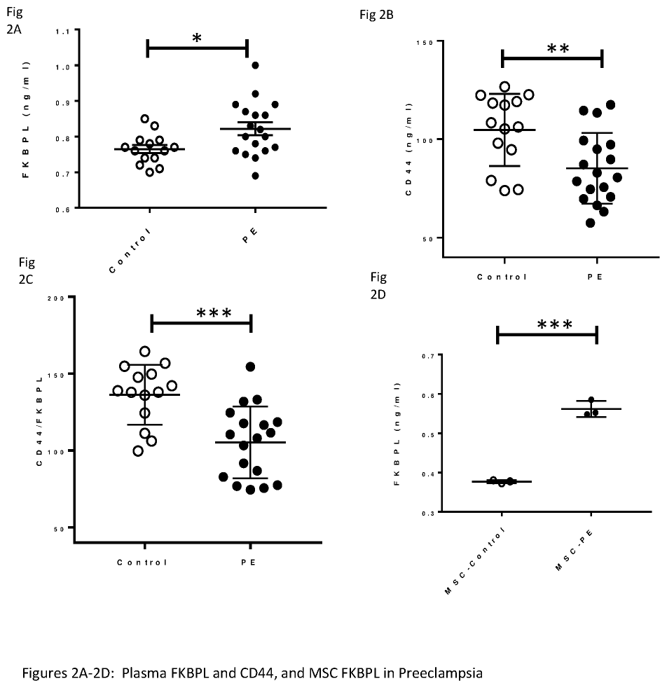

Using a separate cohort of patients, plasma FKBPL and CD44 levels (n=18, preeclampsia; n=14, control) as well as secreted FKBPL from MSCs isolated from pregnant women with preeclampsia (n=3) and normotensive controls (n=3) was assayed. In contrast with measurements prior to diagnosis, at 15 and 20 weeks gestation, plasma FKBPL levels were increased in women with diagnosed preeclampsia compared to controls (FIG. 2A; 0.81 ng/ml±0.018 SEM vs 0.76 ng/ml±0.011 SEM, p<0.05) whereas plasma CD44 levels were decreased in the same samples (FIG. 2B; 85.1 ng/ml±4.2 SEM vs 104.7 ng/ml±4.9 SEM, p<0.01). The CD44/FKBPL ratio was also significantly reduced in preeclampsia cases compared to controls (FIG. 2C; 105.3 ng/ml±5.5 SEM vs 136.3 ng/ml±5.2, p<0.001).

Secreted FKBPL from MSCs cultured from adipose tissue of pregnant women undergoing C-section was higher in cells cultured from women post-preeclampsia diagnosis, compared to healthy pregnant controls (FIG. 2D).

TABLE 4
is a representation of trends in CD44
and FKBPL during onset of preeclampsia:

| | Post-Preeclampsia Diagnosis | | |
|---|---|---|---|
| Variable | Cases | Controls | p-value |
| CD44 (ng/ml) | 85.1 ± 4.2 | 104.7 ± 4.9 | p < 0.05 |
| FKBPL (ng/ml) | 0.81 ± 0.018 | 0.76 ± 0.011 | p < 0.01 |
| CD44/FKBPL | 105.3 ± 5.5 | 136.3 ± 5.2 | p < 0.001 |
| ratio | | | |

Taken together, these data suggest that serum FKBPL and CD44 can be effective biomarkers not only for determining risk, but also for monitoring women at risk for preeclampsia, and for determining options for management of pregnancies following diagnosis.

### Example III: Endothelial and Trophoblast Cell Angiogenic Function is Improved Following Exposure to Hypoxia or Treatment with MSCs in Association with Reduced FKBPL and Increased CD44 Levels

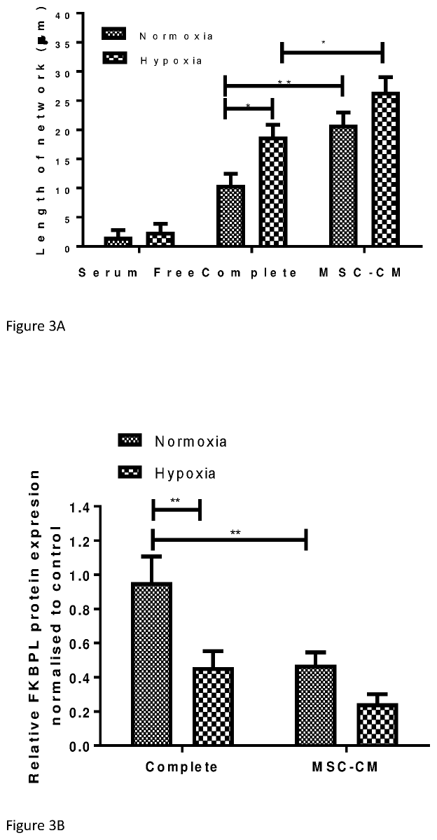

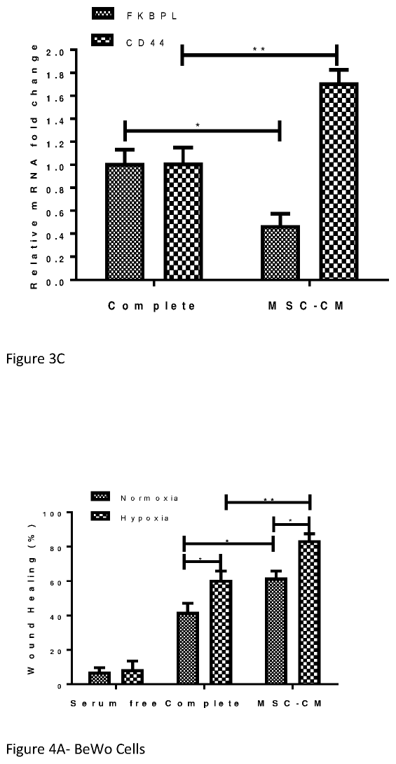

The influence of hypoxia and MSC conditioned medium (MSC-CM) on endothelial cell tubule formation and trophoblast migration, as well as the effect on FKBPL signalling by trophoblasts was investigated. In the tubule formation assay, an increase in the length of the tubule network of the HUVECs was observed between normoxia and hypoxia (p=0.014, n=6) in complete/normal medium. MSC-CM treatment also led to an increase in the length of the tubule networks of HUVECs compared to complete medium both in normoxic (p=0.002, n=6) and hypoxic conditions (p=0.02, n=6; FIG. 3A), 6 h after seeding the cells in Matrigel. Overall, improved angiogenic properties, as a result of hypoxia or MSC-CM, were associated with reduced FKBPL protein levels following exposure to hypoxia in complete medium conditions (p<0.01, n=6), and between complete medium and MSC-CM conditions in normoxia (p<0.01, n=6; FIG. 3B). In hypoxia, at the mRNA level, FKBPL was reduced (p=0.021, n=3) and CD44 concomitantly increased (p=0.005, n=3) as a result of MSC-CM treatment of HUVECs (FIG. 3C).

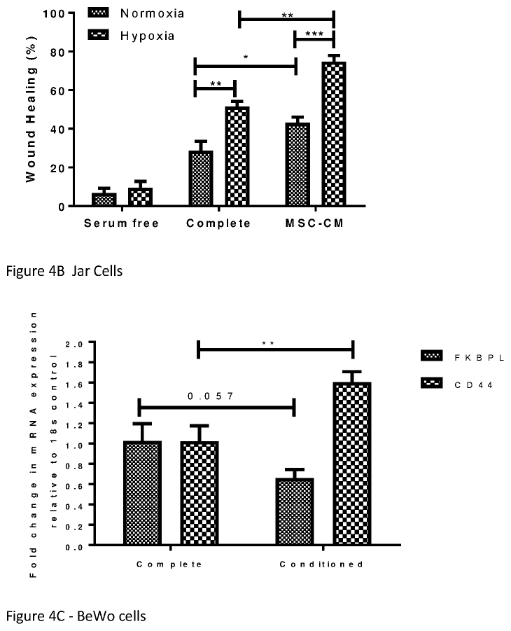

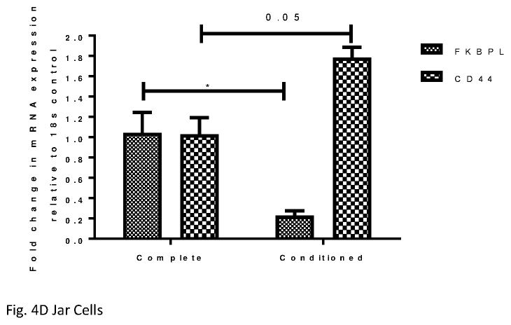

When trophoblast cells, BeWo and Jar, were exposed to hypoxia and/or MSC-CM, an increase in cell migration was also observed in both complete medium and MSC-CM as a result of hypoxia [BeWo: p=0.03 (complete; n=3), p=0.01 (MSC-CM, n=3); Jar: p=0.002 (complete, n=3), p<0.001 (MSC-CM, n=3); FIG. 4A&B], and MSCs in normoxia (BeWo: p=0.02, n=3; Jar: p=0.04, n=3; FIG. 4A&B) or hypoxia (BeWo: p=0.008, n=3; Jar: p=0.001, n=3; FIGS. 4A&B). A similar trend indicating downregulation of FKBPL and upregulations of CD44 mRNA expression was observed in BeWo and Jar cells as in HUVECs cells (FIGS. 4C&D).

Taken together, these data suggest that FKBPL and CD44 are involved in mediating the pro-angiogenic effect of hypoxia or MSC-CM on both endothelial and trophoblast cells.

SEQUENCE LISTINGS:
Preeclampsia Biomarkers and Use Thereof

SEQ ID NO 1
<211> 1342
<212> DNA
<213> Homo sapiens
gttagttgtc agtatctttc ccagttgttc cgccccctac ccccgcctcc cgcaccgcgc ccctctccgg ctgccctctc
cgcgtggggc aaggctccga gggcagcatt cagtagccat ttagctttgg aaggagaggt gattcgaatg gcccggctcc
tcctgtcacc atgctaggca ctttggccgc gcaggtgctg acctgaacct ggttcatccc tttctgacca aaactgttca
ctcaccgtgg aagggactaa gcatccatat ggagacgcca ccagtcaata caattggaga aaaggacacc
tctcagccgc aacaagagtg ggaaaagaac cttcgggaga accttgattc agttattcag attaggcagc agccccgaga
ccctcctacc gaaacgcttg agctggaagt aagcccagat ccagccagcc aaattctaga gcatactcaa ggagctgaaa
aactggttgc tgaacttgaa ggagactctc ataagtctca tggatcaacc agtcagatgc cagaggccct tcaagcttct
gatctctggt actgccccga tgggagcttt gtcaagaaga tcgtaatccg tggccatggc ttggacaaac ccaaactagg
ctcctgctgc cgggtactgg ctttggggtt tcctttcgga tcagggccgc cagagggctg gacagagcta actatgggcg
tagggccatg gagggaggaa acttgggggg agctcataga gaaatgcttg gagtccatgt gtcaaggtga
ggaagcagag cttcagctgc ctgggcactc tggacctcct gtcaggctca cactggcatc cttcactcaa ggccgagact
cctgggagct ggagactagc gagaaggaag ccctggccag ggaagaacgt gcaaggggca cagaactatt
tcgagctggg aaccctgaag gagctgcccg atgctatgga cgggctcttc ggctgctcct gactttaccc ccacctggcc
ctccagaacg aactgtcctt catgccaatc tggctgcctg tcagttgttg ctagggcagc ctcagttggc agcccagagc
tgtgaccggg tgttggagcg ggagcctggc catttaaagg ccttataccg aaggggggtt gcccaggctg cccttgggaa
cctggaaaaa gcaactgctg acctcaagaa ggtgctggcg atagatccca aaaaccgggc agcccaggag
gaactgggga aggtggtcat tcaggggaag aaccaggatg cagggctggc tcagggtctg cgcaagatgt ttggctgatt
aaaagttaaa ccttaaaaga ga
SEQ ID NO 2
<211> 349
<212> PRT
<213> Homosapiens
Met Glu Thr Pro Pro Val Asn Thr He Gly Glu Lys Asp Thr Ser Gln Pro Gln Gln Glu Trp Glu Lys
Asn Leu Arg Glu Asn Leu Asp Ser Val Ile Gln Ile Arg Gln Gln Pro Arg Asp Pro Pro Thr Glu Thr
Leu Glu Leu Glu Val Ser Pro Asp Pro Ala Ser Gln He Leu Glu His Thr Gln Gly Ala Glu Lys Leu
Val Ala Glu Leu Glu Gly Asp Ser His Lys Ser His Gly Ser Thr Ser Gln Met Pro Glu Ala Leu
Gln Ala Ser Asp Leu Trp Tyr Cys Pro Asp Gly Ser Phe Val Lys Lys He Val He Arg Gly His Gly
Leu Asp Lys Pro Lys Leu Gly Ser Cys Cys Arg Val Leu Ala Leu Gly Phe Pro Phe Gly Ser Gly
Pro Pro Glu Gly Trp Thr Glu Leu Thr Met Gly Val Gly Pro Trp Arg Glu Glu Thr Trp Gly Glu Leu
Ile Glu Lys Cys Leu Glu Ser Met Cys Gln Gly Glu Glu Ala Glu Leu Gln Leu Pro Gly His Ser
Gly Pro Pro Val Arg Leu Thr Leu Ala Ser Phe Thr Gln Gly Arg Asp Ser Trp Glu Leu Glu Thr
Ser Glu Lys Glu Ala Leu Ala Arg Glu Glu Arg Ala Arg Gly Thr Glu Leu Phe Arg Ala Gly Asn
Pro Glu Gly Ala Ala Arg Cys Tyr Gly Arg Ala Leu Arg Leu Leu Leu Thr Leu Pro Pro Pro Gly
Pro Pro Glu Arg Thr Val Leu His Ala Asn Leu Ala Ala Cys Gln Leu Leu Leu Gly Gln Pro Gln
Leu Ala Ala Gln Ser Cys Asp Arg Val Leu Glu Arg Glu Pro Gly His Leu Lys Ala Leu Tyr Arg
Arg Gly Val Ala Gln Ala Ala Leu Gly Asn Leu Glu Lys Ala Thr Ala Asp Leu Lys Lys Val Leu
Ala He Asp Pro Lys Asn Arg Ala Ala Gln Glu Glu Leu Gly Lys Val Val Ile Gln Gly Lys Asn Gln
Asp Ala Gly Leu Ala Gln Gly Leu Arg Lys Met Phe Gly
SEQ ID NO 3
<211> 2174
<212> DNA
<213> Homo sapiens
atggagacgc caccagtcaa tacaattgga gaaaaggaca cctctcagcc gcaacaagag tgggaaaaga
accttcggga gaaccttgat tcagttattc agattaggca gcagccccga gaccctccta ccgaaacgct tgagctggaa
gtaagcccag atccagccag ccaaattcta gagcatactc aaggagctga aaaactggtt gctgaacttg aaggagactc
tcataagtct catggatcaa ccagtcagat gccagaggcc cttcaagctt ctgatctctg gtactgcccc gatgggagct
ttgtcaagaa gatcgtaatc cgtggccatg gcttggacaa acccaaacta ggctcctgct gccgggtact ggctttgggg
tttcctttcg gatcagggcc gccagagggc tggacagagc taactatggg cgtagggcca tggagggagg aaacttgggg
ggagctcata gagaaatgct tggagtccat gtgtcaaggt gaggaagcag agcttcagct gcctgggcac actggacctc
ctgtcgggct cacactggca tccttcactc aaggccgaga ctcctgggag ctggagacta gcgagaagga agccctggcc
agggaagaac gtgcaagggg cacagaacta tttcgagctg ggaaccctga aggagctgcc cgatgctatg
gacgggctct tcggctgctc ctgactttac ccccacctgg ccctccagaa cgaactgtcc ttcatgccaa tctggctgcc
tgtcagttgt tgctagggca gcctcagttg gcagcccaga gctgtgaccg ggtgttggag cgggagcctg gccatttaaa
ggccttatac cgaagggggg ttgcccaggc tgcccttggg aacctggaaa aagcaactgc tgacctcaag aaggtgctgg
cgatagatcc caaaaaccgg gcagcccagg aggaactggg gaaggtggtc attcagggga agaaccagga
tgcagggctg gctcagggtc tgcgcaagat gtttggctga ttaaaagtta aaccttaaaa gagaaaaaaa aaaaaaaatg
gagacgccac cagtcaatac aattggagaa aaggacacct ctcagccgca acaagagtgg gaaaagaacc
ttcgggagaa ccttgattca gttattcaga ttaggcagca gccccgagac cctcctaccg aaacgcttga gctggaagta
agcccagatc cagccagcca aattctagag catactcaag gagctgaaaa actggttgct gaacttgaag gagactctca
taagtctcat ggatcaacca gtcagatgcc agaggccctt caagcttctg atctctggta ctgccccgat gggagctttg
tcaagaagat cgtaatccgt ggccatggct tggacaaacc caaactaggc tcctgctgcc gggtactggc tttggggttt
cctttcggat cagggccgcc agagggctgg acagagctaa ctatgggcgt agggccatgg agggaggaaa
cttgggggga gctcatagag aaatgcttgg agtccatgtg tcaaggtgag gaagcagagc ttcagctgcc tgggcacact
ggacctcctg tcgggctcac actggcatcc ttcactcaag gccgagactc ctgggagctg gagactagcg agaaggaagc
cctggccagg gaagaacgtg caaggggcac agaactattt cgagctggga accctgaagg agctgcccga
tgctatggac gggctcttcg gctgctcctg actttacccc cacctggccc tccagaacga actgtccttc atgccaatct
ggctgcctgt cagttgttgc tagggcagcc tcagttggca gcccagagct gtgaccgggt gttggagcgg gagcctggcc
atttaaaggc cttataccga aggggggttg cccaggctgc ccttgggaac ctggaaaaag caactgctga cctcaagaag
gtgctggcga tagatcccaa aaaccgggca gcccaggagg aactggggaa ggtggtcatt caggggaaga
accaggatgc agggctggct cagggtctgc gcaagatgtt tggctgatta aaagttaaac cttaaaagag aaaaaaaaaa
aaaa
SEQ ID NO 4
[00242]<211> 349
[00243]<212> PRT
[00244]<213> Homo sapiens
Met Glu Thr Pro Pro Val Asn Thr He Gly Glu Lys Asp Thr Ser Gln Pro Gln Gln Glu Trp Glu Lys
Asn Leu Arg Glu Asn Leu Asp Ser Val Ile Gln Ile Arg Gln Gln Pro Arg Asp Pro Pro Thr Glu Thr
Leu Glu Leu Glu Val Ser Pro Asp Pro Ala Ser Gln He Leu Glu His Thr Gln Gly Ala Glu Lys Leu
Val Ala Glu Leu Glu Gly Asp Ser His Lys Ser His Gly Ser Thr Ser Gln Met Pro Glu Ala Leu
Gln Ala Ser Asp Leu Trp Tyr Cys Pro Asp Gly Ser Phe Val Lys Lys He Val He Arg Gly His Gly
Leu Asp Lys Pro Lys Leu Gly Ser Cys Cys Arg Val Leu Ala Leu Gly Phe Pro Phe Gly Ser Gly
Pro Pro Glu Gly Trp Thr Glu Leu Thr Met Gly Val Gly Pro Trp Arg Glu Glu Thr Trp Gly Glu Leu
Ile Glu Lys Cys Leu Glu Ser Met Cys Gln Gly Glu Glu Ala Glu Leu Gln Leu Pro Gly His Thr
Gly Pro Pro Val Gly Leu Thr Leu Ala Ser Phe Thr Gln Gly Arg Asp Ser Trp Glu Leu Glu Thr
Ser Glu Lys Glu Ala Leu Ala Arg Glu Glu Arg Ala Arg Gly Thr Glu Leu Phe Arg Ala Gly Asn
Pro Glu Gly Ala Ala Arg Cys Tyr Gly Arg Ala Leu Arg Leu Leu Leu Thr Leu Pro Pro Pro Gly
Pro Pro Glu Arg Thr Val Leu His Ala Asn Leu Ala Ala Cys Gln Leu Leu Leu Gly Gln Pro Gln
Leu Ala Ala Gln Ser Cys Asp Arg Val Leu Glu Arg Glu Pro Gly His Leu Lys Ala Leu Tyr Arg
Arg Gly Val Ala Gln Ala Ala Leu Gly Asn Leu Glu Lys Ala Thr Ala Asp Leu Lys Lys Val Leu
Ala He Asp Pro Lys Asn Arg Ala Ala Gln Glu Glu Leu Gly Lys Val Val Ile Gln Gly Lys Asn Gln
Asp Ala Gly Leu Ala Gln Gly Leu Arg Lys Met Phe Gly
SEQ ID NO 5
<211> 15
<212> PRT
<213> Artificial Sequence
<223> Biotin lygase tag amino acid sequence
Leu His His He Leu Asp Ala Gln Lys Met Val Trp Asn His Arg

What is claimed is:

1. A method of determining risk for developing preeclampsia in a pregnant female human subject, the method comprising detecting the level of CD44 in a biological sample of the subject using an agent binding CD44, wherein a level of the CD44 above a predetermined threshold in the sample is indicative of an increased risk for the subject to develop preeclampsia at or after gestational week 30.

2. The method of claim 1, wherein the sample is of a gestation week 20 and on.

3. The method of claim 1, further comprising repeating the detecting at least once in a sample from a later gestational week and comparing with earlier CD44 level, wherein an increase in the level of CD44 in the later detection relative to the earlier CD44 level is indicative of an increased risk for the subject to develop preeclampsia at or after gestational week 30, and wherein the later gestational week is no later than gestational week 30.

4. A method of determining risk for developing preeclampsia in a pregnant female human subject, the method comprising:
a) detecting the level of CD44 and the level of FKBPL in a biological sample of the subject using an agent binding CD44 and an agent binding FKBPL,
b) determining the ratio of CD44/FKBPL in the sample,
c) wherein a ratio of CD44/FKBPL above a predetermined threshold in the sample is indicative of an increased risk for the subject to develop preeclampsia at or after gestational week 30.

5. The method of claim 4, wherein the sample is of gestational weeks 15-30.

6. The method of claim 4, further comprising repeating the detecting the level of CD44 and FKBPL and determining the CD44/FKBPL ratio at least once in a sample from a later gestational week and comparing with earlier CD44/FKBPL ratio, wherein an increase in the CD44/FKBPL ratio in the later detection relative to the earlier CD44/FKBPL ratio is indicative of an increased risk for the subject to develop preeclampsia at or after gestational week 30, and wherein the later gestational week is no later than gestational week 30.

7. The method of any one of claims 4-6, further comprising determining mean arterial blood pressure in the subject, and wherein elevated MABP and a ratio of CD44/FKBPL above a predetermined threshold in the sample is indicative of an increased risk for the subject to develop preeclampsia at or after gestational week 30.

8. A method for managing pregnancy in a pregnant female human subject at gestational week 20-30, the method comprising:
a) detecting the level of CD44 in a biological sample of the subject using an agent binding CD44,
b) wherein a level of the CD44 is above a predetermined threshold in the sample, referring the subject for high risk pregnancy management.

9. A method for managing pregnancy in a pregnant female human subject at gestational week 15-30, the method comprising:
a) detecting the level of CD44 and the level of FKBPL in a biological sample of the subject using an agent binding CD44 and an agent binding FKBPL,
b) determining the ratio of CD44/FKBPL in the sample,
c) wherein a ratio of CD44/FKBPL is above a predetermined threshold in the sample, referring the subject for high risk pregnancy management.

10. The method of claim 9, further comprising determining mean arterial blood pressure in the subject, and referring the subject for high-risk pregnancy management when mean arterial blood pressure is elevated and the ratio of CD44/FKBPL is above the predetermined threshold.

11. The method of any one of claims 8-10, wherein the high risk pregnancy management comprises monitoring blood pressure and proteinuria in the subject.

12. The method of any one of claims 1-11, wherein the subject exhibits normal values for at least one indicator of preeclampsia selected from the group consisting of blood pressure, proteinuria and liver function.

13. A method of treating a pregnant female human subject for preeclampsia, the method comprising:
a) detecting the level of CD44 in a biological sample of the subject using an agent binding CD44, wherein the biological sample is of gestational week 20-30,
b) wherein a level of the CD44 in the biological sample is above a predetermined threshold, testing the subject for at least one indicator of preeclampsia, and
c) wherein the at least one indicator of preeclampsia is positive, treating the subject for preeclampsia at or after gestational week 30.

14. The method of claim 13, wherein step (a) further comprises determining mean arterial blood pressure in the subject, and step (b) comprises testing the subject wherein levels of the CD44 and the mean arterial blood pressure are above a predetermined threshold, and wherein the at least one indicator of preeclampsia is not mean arterial blood pressure.

15. A method of treating a pregnant female human subject for preeclampsia, the method comprising:
a) detecting the level of CD44 and a level of FKBPL in a biological sample of the subject using an agent binding CD44 and an agent binding FKBPL, wherein the biological sample is of gestational week 15-30,
b) wherein a ratio of the CD44/FKBPL levels in the biological sample is above a predetermined threshold, testing the subject for at least one indicator of preeclampsia, and
c) wherein the at least one indicator of preeclampsia is positive, treating the subject for preeclampsia at or after gestational week 30.

16. A method of treating a pregnant female human subject for preeclampsia, the method comprising:
a. detecting the level of CD44 and a level of FKBPL in a first biological sample of the subject using an agent binding CD44 and an agent binding FKBPL, wherein the first biological sample is of gestational week 15-30,
b) detecting the level of CD44 and a level of FKBPL in a second biological sample of the subject using an agent binding CD44 and an agent binding FKBPL, wherein the second biological sample is of gestational week later than gestational week 30,
c) wherein a ratio of the CD44/FKBPL levels in the first biological sample is above a first predetermined threshold and a ratio of the CD44/FKBPL levels in the second biological sample is below a second predetermined threshold, testing the subject for at least one indicator of preeclampsia, and
d) wherein the at least one indicator of preeclampsia is positive, treating the subject for preeclampsia.

17. The method of claim 16, wherein step (a) further comprises determining mean arterial blood pressure in the subject, and step (b) comprises testing the subject wherein the ratio of the CD44/FKBPL levels and the mean arterial blood pressure are above a predetermined threshold, and wherein the at least one indicator of preeclampsia is not mean arterial blood pressure.

18. The method of claim 16 or 17, wherein the at least one indicator is selected from the group consisting of hypertension, proteinuria, low platelet count, impaired liver function, signs of kidney problems (other than proteinuria), pulmonary edema and headaches or visual disturbances.

19. The method of any one of claim 17 or 18, wherein the preeclampsia treatment is selected from the group consisting of anti-hypertensive treatment, steroids and seizure prophylaxis.

20. A method of treating a pregnant female human subject for preeclampsia, the method comprising:
a) detecting the level of CD44 in a biological sample of the subject using an agent binding CD44, wherein the biological sample is of gestational week later than gestational week 30,
b) wherein a level of the CD44 in the biological sample is below a predetermined threshold, testing the subject for at least one indicator of preeclampsia, and
c) wherein the at least one indicator of preeclampsia is positive, treating the subject for preeclampsia.

21. The method of claim 20, wherein the at least one indicator of preeclampsia is mean arterial blood pressure.

22. A method of treating a pregnant female human subject for preeclampsia, the method comprising:
a) detecting the level of CD44 and a level of FKBPL in a biological sample of the subject using an agent binding CD44 and an agent binding FKBPL, wherein the biological sample is of a gestational week later than gestational week 30,
b) wherein a ratio of the CD44/FKBPL levels in the biological sample is below a predetermined threshold, testing the subject for at least one indicator of preeclampsia, and
c) wherein the at least one indicator of preeclampsia is positive, treating the subject for preeclampsia.

23. The method of claim 22, wherein the at least one indicator is selected from the group consisting of hypertension, proteinuria, platelet count, impaired liver function, signs of kidney problems (other than proteinuria), pulmonary edema and headaches or visual disturbances.

24. The method of claim 22, wherein the at least one indicator of preeclampsia is not MABP.

25. The method of any one of claims 20-24, wherein the preeclampsia treatment is selected from the group consisting of anti-hypertensive treatment, steroids and seizure prophylaxis.

26. The method of any one of claims 1-24, wherein the biological sample is whole blood or an isolated blood fraction.

27. The method of claim 26, wherein the isolated blood fraction is serum or plasma.

28. The method of any one of claims 1-27, wherein the agent binding CD44 is an antibody specifically binding CD44.

29. The method of any one of claims 4-7, 9-11 and 14-27, wherein the agent binding FKBPL is an antibody specifically binding FKBPL.

30. The method of any one of claim 28 or 29, wherein the antibody is a monoclonal antibody.

31. The method of any one of claims 1-30, wherein the predetermined threshold is a level of the CD44 or the ratio of CD44/FKBPL in normal, healthy pregnant gestational age-matched females.

32. A composition-of-matter comprising a biological sample of a pregnant female human subject comprising an agent binding CD44 and an agent binding FKBPL.

33. A composition of matter comprising:
a) a first portion of a biological sample of a pregnant female human subject comprising an agent binding CD44, and
b) a second portion of the biological sample of the pregnant female human subject comprising an agent binding FKBPL.

34. The composition-of-matter of claim 32 or 33, wherein the biological sample is whole blood or an isolated blood fraction.

35. The composition-of-matter of claim 34, wherein the isolated blood fraction is plasma or serum.

36. The composition-of-matter of any one of claims 32-35, wherein the sample is of a gestation week of 15 weeks and on.

37. The composition-of-matter of any one of claims 32-36, wherein the agent binding CD44 is an antibody specifically binding CD44.

38. The composition-of-matter of any one of claims 32-36, wherein the agent binding FKBPL is an antibody specifically binding FKBPL.

39. The composition-of-matter of any one of claim 37 or 38, wherein the antibody is a monoclonal antibody.

40. A diagnostic kit for use in diagnosing preeclampsia or risk to develop preeclampsia in a pregnant female human subject, the kit comprising: (a) a first agent which specifically binds soluble CD44, wherein the first agent is detectably labeled or coupled to an enzyme; (b) a normal reference sample for CD44, wherein the normal reference sample for CD44 is a sample containing a level of CD44 found in a pregnant human female subject not having preeclampsia or risk to develop preeclampsia; a pregnant human female subject not having early-onset preeclampsia or eclampsia; (c) a second agent which binds soluble FK 506 Binding Protein Like (FKBPL) wherein the second agent is detectably labeled or coupled to an enzyme and (d) a normal reference sample for FKBPL, wherein the normal reference for FKBPL sample is a sample containing a level of FKBPL found in a pregnant human female subject not having preeclampsia or risk to develop preeclampsia; a pregnant human female subject not having early onset preeclampsia.

41. The kit of claim 40, wherein the normal reference sample of CD44 contains CD44 in a range of concentrations between 20 and 300 ng/ml.

42. The kit of claims 40 and 41, wherein the normal reference sample of FKBPL contains FKBPL in a range of concentrations between 0.1 and 5 ng/ml.

43. The kit of any one of claims 40-42, wherein the agent binding FKBPL is an antibody, or FKBPL-binding fragment thereof.

44. The kit of any one of claims 40-43, wherein the agent binding CD44 is an antibody, or CD44-binding fragment thereof.

45. The kit of claim 43 or 44, wherein the antibody is a monoclonal antibody.

46. The kit of any one of claims 40-45, wherein the agent binding CD44 and/or the agent binding FKBPL are immobilized in a solid phase.

47. An agent for treatment of preeclampsia for use in a pregnant human female subject exhibiting a level of CD44:
a) greater than a predetermined threshold in a biological sample therefrom, wherein the sample is from 20-30 weeks gestation; or
b) lower than a second predetermined threshold, wherein the sample is from 30 or more weeks gestation.

48. An agent for treatment of preeclampsia for use in a pregnant human female subject exhibiting a ratio of CD44/FKBPL:
a) greater than a predetermined threshold in a biological sample therefrom, wherein the sample is from 15-30 weeks gestation, or
b) lower than a second predetermined threshold wherein the sample is from 30 or more weeks gestation.

49. The agent for treatment of claim 48, wherein the pregnant human female subject further exhibits an elevated MABP.

50. The agent for treatment of any one of claims 47-49, wherein the biological sample is whole blood or an isolated blood fraction.

51. The agent for treatment of claim 50, wherein the isolated blood fraction is plasma or serum.


SEQUENCE LISTING (5 sequences)

SEQ ID NO: 1 - DNA, 1342 bp, Homo sapiens
gttagttgtcagtatctttcccagttgttccgccccctacccccgcctcccgcaccgcgcccctctccggctgccctctccgcgtggggcaaggctccgagggcagcattcagtagccatttagctttggaaggagaggtgattcgaatggcccggctcctcctgtcaccatgctaggcactttggccgcgcaggtgctgacctgaacctggttcatccctttctgaccaaaactgttcactcaccgtggaagggactaagcatccatatggagacgccaccagtcaatacaattggagaaaaggacacctctcagccgcaacaagagtgggaaaagaaccttcgggagaaccttgattcagttattcagattaggcagcagccccgagaccctcctaccgaaacgcttgagctggaagtaagcccagatccagccagccaaattctagagcatactcaaggagctgaaaaactggttgctgaacttgaaggagactctcataagtctcatggatcaaccagtcagatgccagaggcccttcaagcttctgatctctggtactgccccgatgggagctttgtcaagaagatcgtaatccgtggccatggcttggacaaacccaaactaggctcctgctgccgggtactggctttggggtttcctttcggatcagggccgccagagggctggacagagctaactatgggcgtagggccatggagggaggaaacttggggggagctcatagagaaatgcttggagtccatgtgtcaaggtgaggaagcagagcttcagctgcctgggcactctggacctcctgtcaggctcacactggcatccttcactcaaggccgagactcctgggagctggagactagcgagaaggaagccctggccagggaagaacgtgcaaggggcacagaactatttcgagctgggaaccctgaaggagctgcccgatgctatggacgggctcttcggctgctcctgactttacccccacctggccctccagaacgaactgtccttcatgccaatctggctgcctgtcagttgttgctagggcagcctcagttggcagcccagagctgtgaccgggtgttggagcgggagcctggccatttaaaggccttataccgaaggggggttgcccaggctgcccttgggaacctggaaaaagcaactgctgacctcaagaaggtgctggcgatagatcccaaaaaccgggcagcccaggaggaactggggaaggtggtcattcaggggaagaaccaggatgcagggctggctcagggtctgcgcaagatgtttggctgattaaaagttaaaccttaaaagaga

SEQ ID NO: 2 - protein, 349 aa, Homo sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHSGPPVRLTLASFTQGRDSWELETSEKEALAREERARGTELFRAGNPEGAARCYGRALRLLLTLPPPGPPERTVLHANLAACQLLLGQPQLAAQSCDRVLEREPGHLKALYRRGVAQAALGNLEKATADLKKVLAIDPKNRAAQEELGKVVIQGKNQDAGLAQGLRKMFG

SEQ ID NO: 3 - DNA, 2174 bp, Home Sapiens
atggagacgccaccagtcaatacaattggagaaaaggacacctctcagccgcaacaagagtgggaaaagaaccttcgggagaaccttgattcagttattcagattaggcagcagccccgagaccctcctaccgaaacgcttgagctggaagtaagcccagatccagccagccaaattctagagcatactcaaggagctgaaaaactggttgctgaacttgaaggagactctcataagtctcatggatcaaccagtcagatgccagaggcccttcaagcttctgatctctggtactgccccgatgggagctttgtcaagaagatcgtaatccgtggccatggcttggacaaacccaaactaggctcctgctgccgggtactggctttggggtttcctttcggatcagggccgccagagggctggacagagctaactatgggcgtagggccatggagggaggaaacttggggggagctcatagagaaatgcttggagtccatgtgtcaaggtgaggaagcagagcttcagctgcctgggcacactggacctcctgtcgggctcacactggcatccttcactcaaggccgagactcctgggagctggagactagcgagaaggaagccctggccagggaagaacgtgcaaggggcacagaactatttcgagctgggaaccctgaaggagctgcccgatgctatggacgggctcttcggctgctcctgactttacccccacctggccctccagaacgaactgtccttcatgccaatctggctgcctgtcagttgttgctagggcagcctcagttggcagcccagagctgtgaccgggtgttggagcgggagcctggccatttaaaggccttataccgaaggggggttgcccaggctgcccttgggaacctggaaaaagcaactgctgacctcaagaaggtgctggcgatagatcccaaaaaccgggcagcccaggaggaactggggaaggtggtcattcaggggaagaaccaggatgcagggctggctcagggtctgcgcaagatgtttggctgattaaaagttaaaccttaaaagagaaaaaaaaaaaaaaatggagacgccaccagtcaatacaattggagaaaaggacacctctcagccgcaacaagagtgggaaaagaaccttcgggagaaccttgattcagttattcagattaggcagcagccccgagaccctcctaccgaaacgcttgagctggaagtaagcccagatccagccagccaaattctagagcatactcaaggagctgaaaaactggttgctgaacttgaaggagactctcataagtctcatggatcaaccagtcagatgccagaggcccttcaagcttctgatctctggtactgccccgatgggagctttgtcaagaagatcgtaatccgtggccatggcttggacaaacccaaactaggctcctgctgccgggtactggctttggggtttcctttcggatcagggccgccagagggctggacagagctaactatgggcgtagggccatggagggaggaaacttggggggagctcatagagaaatgcttggagtccatgtgtcaaggtgaggaagcagagcttcagctgcctgggcacactggacctcctgtcgggctcacactggcatccttcactcaaggccgagactcctgggagctggagactagcgagaaggaagccctggccagggaagaacgtgcaaggggcacagaactatttcgagctgggaaccctgaaggagctgcccgatgctatggacgggctcttcggctgctcctgactttacccccacctggccctccagaacgaactgtccttcatgccaatctggctgcctgtcagttgttgctagggcagcctcagttggcagcccagagctgtgaccgggtgttggagcgggagcctggccatttaaaggccttataccgaaggggggttgcccaggctgcccttgggaacctggaaaaagcaactgctgacctcaagaaggtgctggcgatagatcccaaaaaccgggcagcccaggaggaactggggaaggtggtcattcaggggaagaaccaggatgcagggctggctcagggtctgcgcaagatgtttggctgattaaaagttaaaccttaaaagagaaaaaaaaaaaaaa

SEQ ID NO: 4 - protein, 349 aa, Home Sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHTGPPVGLTLASFTQGRDSWELETSEKEALAREERARGTELFRAGNPEGAARCYGRALRLLLTLPPPGPPERTVLHANLAACQLLLGQPQLAAQSCDRVLEREPGHLKALYRRGVAQAALGNLEKATADLKKVLAIDPKNRAAQEELGKVVIQGKNQDAGLAQGLRKMFG

SEQ ID NO: 5 - protein, 15 aa, Artificial Sequence
  Biotin lygase tag amino acid sequence
LHHILDAQKMVWNHR